(19) United States
(12) Patent Application Publication
(10) Pub. No.: US 2020/0261427 A1
(43) Pub. Date: Aug. 20, 2020

(54) TOPICAL RAPAMYCIN FORMULATIONS AND THEIR USE IN TREATING FACIAL ANGIOFIBROMAS AND OTHER SKIN DISORDERS

(71) Applicant: Al Therapeutics, Inc., Guilford, CT (US)

(72) Inventors: Henri Lichenstein, Guilford, CT (US); Jonathan M. Rothberg, Guilford, CT (US); Tian Xu, Shanghai (CN); Jeff Grotzke, Guilford, CT (US); Paul Beckett, Yorktown Heights, NY (US); Keith Fandrick, Guilford, CT (US)

(21) Appl. No.: 16/795,052

(22) Filed: Feb. 19, 2020

Related U.S. Application Data
(60) Provisional application No. 62/835,630, filed on Apr. 18, 2019.
(60) Provisional application No. 62/807,786, filed on Feb. 20, 2019.

(51) Int. Cl. A61K 31/436; A61K 9/06; A61K 9/00; A61K 47/38; A61K 47/32; A61K 47/26; A61K 47/10; A61K 9/14

(52) U.S. Cl. CPC A61K 31/436; A61K 9/06; A61K 9/0014; A61K 9/14; A61K 47/32; A61K 47/26; A61K 47/10; A61K 47/38

(57) ABSTRACT

The present disclosure provides gel compositions of rapamycin for topical administration and related compositions and methods including their use in the treatment of a skin condition, disease or disorder.

### FIELD OF THE INVENTION

The present invention relates to topical rapamycin compositions and related methods for treating facial angiofibromas and other skin diseases and disorders.

### BACKGROUND

Patients with skin disorders may experience fearful anticipation of interaction with others and may develop avoidance-coping mechanisms. This may prevent them from partaking fully, or at all, in social and recreational activities or employment. Ultimately, visible symptoms may change how patients see themselves and how they perceive their future. Studies have shown that successful treatment of severe skin disorders, which improves the patient's symptoms and changes their physical appearance, can lead to improvement in psychologic symptoms and a better quality of life.

Tuberous sclerosis complex (TSC) is a genetic disorder caused by mutations in either the TSC1 (hamartin) or TSC2 (tuberin) gene. The TSC1 and TSC2 gene products form a complex inside the cell which acts to inhibit the activity of mammalian target of rapamycin (mTOR). The mTOR signaling pathway stimulates cell proliferation as well as cell survival. In the TSC disease state, either the TSC1 or TSC2 gene product is defective, and the inhibitory complex is unable to form, resulting in unregulated mTOR activity.

TSC is characterized by the growth of numerous noncancerous tumors in many parts of the body. These tumors can occur in the skin, brain, kidneys, and other organs, in some cases leading to significant health problems. Virtually all affected people have skin abnormalities, including patches of unusually light-colored skin, areas of raised and thickened skin, and growths under the nails. Tumors on the face called facial angiofibromas and occur in more than 90% of patients diagnosed with TSC. They begin to appear at around 3 to 4 years of age, do not improve spontaneously and can lead to facial disfigurement if left untreated.

Current treatments for facial angiofibromas include vascular lasers, chemical peels, dermabrasion, and electrodesiccation, among others. While these treatments are somewhat effective at improving the facial appearance, the result is often less than satisfactory. In addition, these procedures are costly, uncomfortable, and often need to be repeated periodically to prevent recurrence.

Rapamycin, a macrolide antibiotic, is also referred to as “sirolimus.” Rapamycin and its derivatives are further described in Nishimura, T. et al. (2001) Am. J. Respir. Crit. Care Med. 163:498-502 and in U.S. Pat. Nos. 6,384,046 and 6,258,823. In the United States, rapamycin/sirolimus has been FDA approved since 1999 and marketed for the prophylaxis of organ rejection and renal transplantation under the trade name RAPAMUNE® by Wyeth (Pfizer). RAPAMUNE® is available in the form of an oral solution (1 mg/ml) or tablet (multiple strengths). Rapamycin/sirolimus was further approved in May 2015 for the treatment of lymphangioleiomyomatosis (LAM Therap.).

US 2010/0305150 (Novartis) describes rapamycin derivatives for treating and preventing neurocutaneous disorders, such as those mediated by TSC including tuberous sclerosis, as well as those mediated by neurofibromatosis type 1 (NF-1).

U.S. Pat. No. 7,416,724 (Regents of the University of Michigan) describes methods of treating a subject with tuberous sclerosis comprising administering to said subject an effective amount of rapamycin.

U.S. Pat. No. 6,958,153 (Wyeth) describes treating skin disorders using macrocyclic lactone antibiotics or an immunosuppressive macrolide, including rapamycin formulated in a topical composition and showing efficacy in human subjects at high concentrations of rapamycin (2.2% to 8%).

US 2012/0022095 (Innova Dermaceuticals, also published as US 2013/0225630 and US 2013/0225631) describes methods of treating facial angiofibromas and cutaneous vascular lesions generally with a topical rapamycin composition comprising from 0.1% to 2% by weight of rapamycin. Following 6 weeks of topical administration to the face using a 1% rapamycin ointment (twice daily) a subject suffering from TSC showed reduced erythema and improved skin texture. Blood serum rapamycin levels did not reach the level of detection (reference range 4-20 ng/ml).

WO 2012/142145 A1 (Dow Pharmaceutical Sciences) describes treating a skin condition exhibiting telangiectasia, a dilation of blood vessels usually associated with inflammation in the facial region, with rapamycin. Exemplary conditions potentially treatable include rosacea and other skin disorders such as keratosis pilaris, angiofibroma, port wine stain.

WO 2018031789 A1 (The Board of Regents of the University of Texas System) describes topical rapamycin compositions (0.1% to 5% by weight, a liquid glycol, and a dermatologically acceptable carrier) for treating facial angiofibromas or other skin lesions.

A review published in 2015 analyzed the then current data on the use of topical rapamycin in treating facial angiofibromas in TSC. Balestri et al., J. Eur. Acad. Derm. Venereology (2015), 29(1), 14-20. Sixteen reports involving a total of 84 patients were considered, and among these, an improvement of the lesions was reported in 94% of patients. This study notes that several different formulations, e.g., ointments, gels, solutions, and creams were used, over a range of rapamycin concentrations from 0.003% to 1%.

One early trial investigated the safety and efficacy of low dose (0.003% or 0.015%) topical rapamycin for treating facial angiofibromas in patients with TSC. Koenig et al., Drugs in R&D (2012), 12(3), 121-126. This was a small study (23 subjects) in which efficacy was assessed based on a subjective measure, namely the patient's self-reported assessment of whether or not treatment improved their condition, made it worse, or had no effect. Using this measure, less than (but almost) half of the patients in the combined treatment arms reported improvement. The authors note that the results did not reach statistical significance, meaning that a treatment effect of the vehicle alone could not be ruled out.

Truchuelo et al. Derm. Online J. (2012) 8:15 describe treating facial angiofibromas due to TSC with a 1% topical rapamycin preparation in a cream base with rapid improvement in angiofibroma size and density.

Bougueon et al. (2016) describe solubilized rapamycin (0.1%) in Transcutol® (diethylene glycol monoethyl ether) formulated as a cream and used to treat angiofibromas in TSC patients. Bougueon et al. Int. J. Pharma. (2016) 509(1), 279-284.

Although considerable effort has been made to develop an effective topical rapamycin formulation for treating facial angiofibromas and other skin diseases and disorders, there remains a need for improved topical formulations that stabilize rapamycin chemically, against non-enzymatic oxidative degradation, and physically, against crystal formation and growth up to a year or more at room temperature without the need for cooling or refrigeration. In addition, there is a need to provide this physiochemical stability while also increasing delivery of the rapamycin to the dermis of the skin, which is the target area for treatment of angiofibromas and other skin diseases and disorders. The present disclosure provides topical formulations of rapamycin in the form of gel compositions that address these needs.

### SUMMARY

The present disclosure provides chemically and physically stable topical rapamycin compositions and methods for their use in treating a skin condition, disease, or disorder, preferably for treating facial angiofibromas and other skin lesions associated with aberrant activation of mTOR signaling. The chemically and physically stable topical rapamycin compositions described here are also advantageously free of irritating alcohols such as ethanol and isopropanol, providing a less irritating topical composition compared to those containing such excipients. Further, the topical rapamycin compositions described here efficiently deliver a therapeutically relevant amount of rapamycin to the target layer of the skin, the dermis.

In embodiments, the disclosure provides a gel composition for topical administration consisting of a stable suspension of rapamycin in a homogeneous mixture of a gel structure forming base, a solvent, an antioxidant, a buffering agent adapted to maintain the pH of the composition at less than or equal to pH 6, and one or more optional excipients selected from a surfactant, a humectant, a chelating agent, and a preservative.

In embodiments, the gel structure forming base is selected from hydroxyethyl cellulose (HEC) and poly(acrylic acid) (PAA). In accordance with any of the embodiments described here, the poly(acrylic acid) may be crosslinked poly(acrylic acid). In embodiments where the gel structure forming base is HEC, the HEC may be present in an amount of from 0.5 to 5% w/w, preferably from about 1-2% w/w, or 1-1.75% w/w, based on the total weight of the composition. In embodiments where the gel structure forming base is PAA, the PAA may be present in an amount of from about 0.1 to 3% w/w, 0.1 to 2.25% w/w, or 0.25 to 0.75% w/w, based on the total weight of the composition.

In embodiments, the solvent is selected from propylene glycol (PG), dimethyl isosorbide (DMI), and diethylene glycol monoethylether, which may also be referred to by its tradename, Transcutol® or TC. In embodiments, the PG present in an amount of from about 5-25% w/w, preferably about 10-15% w/w, based on the total weight of the composition. In embodiments, the DMI or TC is present in an amount of from about 5-25% w/w, preferably 6-8% w/w, based on the total weight of the composition.

In accordance with any of the foregoing embodiments, a preferred antioxidant is butylated hydroxyanisol (BHA).

In accordance with any of the foregoing embodiments, the composition may further comprise a surfactant. In embodiments, the surfactant is selected from the group consisting of polysorbate 80, polysorbate 60, polysorbate 40, polysorbate 20, PEG-40 stearate, steareth-20, steareth-100, ceteth-20, ceteareth-20, and sodium lauryl sulfate, preferably polysorbate 80. The surfactant may be present in an amount of from about 0.005 to 1% w/w, preferably 0.01 to 0.10% w/w, based on the total weight of the composition.

In accordance with any of the foregoing embodiments, the composition may further comprise a preservative. In embodiments, the preservative is benzyl alcohol. In embodiments, the benzyl alcohol is present in an amount of from about 0.5% to 3% w/w, preferably 0.5% to 1.5% w/w, based on the total weight of the composition.

In accordance with any of the foregoing embodiments, the rapamycin is micronized rapamycin. In embodiments, the micronized rapamycin consists of micronized particles of rapamycin having a particle size distribution (PSD) defined by a D50 in the range of 1-5 microns or 2-3 microns. In further aspects, the PSD is further defined by a D10 in the range of 1-2 microns or 1.2-1.5 microns and a D90 in the range of 4-8 microns. The micronized particles of rapamycin may be present in an amount of from about 0.05% w/w to 2.0% w/w. In some embodiments, the micronized particles of rapamycin are present in an amount of from about 0.1% w/w, 0.3% w/w, 1.0% w/w or 2.0% w/w, based on the total weight of the composition.

In embodiments, the composition comprises hydroxyethyl cellulose (HEC) as the gel structure forming base, dimethyl isosorbide (DMI) as the solvent, and butylated hydroxyanisol (BHA) as the antioxidant, wherein the rapamycin of the composition is stable against chemical degradation and physically stable against crystal growth for at least three months at 5 C.

In embodiments, the composition comprises hydroxyethyl cellulose (HEC) as the gel structure forming base, TC as the solvent, and butylated hydroxyanisol (BHA) as the antioxidant, wherein the rapamycin of the composition is stable against chemical degradation and physically stable against crystal growth for at least three months at 5 C.

In embodiments, the composition comprises hydroxyethyl cellulose (HEC) as the gel structure forming base, propylene glycol (PG) as the solvent, and butylated hydroxyanisol (BHA) as the antioxidant, wherein the rapamycin of the composition is stable against chemical degradation and physically stable against crystal growth for at least three months at 5 C, 25 C or 40 C and for at least six months at 5 C or 25 C.

In embodiments, the composition comprises poly(acrylic acid) as the gel structure forming base, dimethyl isosorbide (DMI) as the solvent, and butylated hydroxyanisol (BHA) as the antioxidant, wherein the rapamycin of the composition is stable against chemical degradation and physically stable against crystal growth for at least three months at 5 C or for at least one month at 25 C or 40 C.

In embodiments, the composition comprises poly(acrylic acid) as the gel structure forming base, diethylene glycol monoethylether (TC) as the solvent, and butylated hydroxyanisol (BHA) as the antioxidant, wherein the rapamycin of the composition is stable against chemical degradation and physically stable against crystal growth for at least three months at 5 C or for at least one month at 25 C or 40 C.

In embodiments, the composition comprises poly(acrylic acid) as the gel structure forming base, propylene glycol as the solvent, and butylated hydroxyanisol (BHA) as the antioxidant, wherein the rapamycin of the composition is stable against chemical degradation and physically stable against crystal growth for at least three months at 5 C or for at least one month at 25 C or 40 C.

In accordance with any of the foregoing embodiments, the composition may further comprise one or both of a surfactant, preferably about 0.025 to 0.25% w/w polysorbate 80, and a preservative, preferably about 0.5 to 3.0% w/w benzyl alcohol.

Preferably, in accordance with any of the foregoing embodiments, the pH of the composition is preferably less than 7.0, most preferably in the range of pH 3-6.

The disclosure also provides the topical rapamycin composition of any of the foregoing embodiments, for use in therapy.

The disclosure also provides the topical rapamycin composition of any of the foregoing embodiments, for use in a method of treating a skin condition, disease, or disorder. In embodiments, the skin condition, disease or disorder is selected from an angiofibroma, hemangioma, a vascular malformation, a pyogenic granuloma, essential telangiectasias, familial multiple discoid fibroma, and cherry angioma. In embodiments, the skin condition, disease or disorder is a facial angiofibroma.

The disclosure also provides the topical rapamycin composition of any of the foregoing embodiments, for use in a method of treating a skin condition, disease, or disorder selected from the group consisting of Acanthosis nigricans, acne, actinic keratosis, allergic conjunctivitis, ameloonychohypohidrotic syndrome, angiokeratoma, angiokeratomas in Fabry disease, angiomas including cherry angioma, senile angioma, spider angioma, strawberry angioma, and tufted angioma, athlete's foot, atopic dermatitis, bacterial vaginosis, balanitis, Bannayan-Riley-Ruvalcaba Syndrome, basal cell carcinoma, basal cell nevus Syndrome, Birt-Hogg-Dube Syndrome, blisters, blue rubber bleb nevus syndrome, bromhidrosis, Brook-Speigler Syndrome, bullous pemphigoid, calluses, candidiasis, carbunculosis, cavernous lymphangioma, cellulitis, cerebral atrophy-associated skin conditions, chelitis granulomatosis, Conradi-Eltinermann disease, Corneodermatoosseous syndrome-associated skin conditions, Cowden disease, cutaneous Castleman disease, cutaneous larva migrans, cutaneous sarcoidosis, cutaneous T-cell lymphoma (CTCL), decubitous ulcer, dermal atrophy incident to aging or senescence, dermatitis including contact dermatitis, drug-induced dermatitis, allergic dermatitis, nummular dermatitis, perioral dermatitis, neurodermatitis, seborrheic dermatitis, and atopic dermatitis, dermatofibrosarcoma protruberans, dermatophytosis, diffuse microcystic lymphatic malformations, discoid lupus erythematosus, dyshydrotic eczema, dyskeratosis congenita, ecthyma, eczema, epidermodysplasia verruciformis, epidermolysis bullosa simplex, epidermolytic ichthyosis, epithelial nevus including verrucous nevus, systematized nevus, inflammatory linear verrucous epidermal nevus, and sebaceous nevus, erysipalus, erythema multiforme, erythrokeratoderma variabilis, extramammary Paget's disease, familial cylindromatosis, familial multiple discoid fibromas, filariasis, focal acral hyperkeratosis, follicular hyperkeratosis, follicular hyperkeratosis associated with pilodental dysplasia with refractive errors, furunculosis, genital warts, gingival hypertrophy, granuloma, Hailey-Hailey disease, hemangioma simplex, hereditary footpad hyperkeratosis as afflicting dogs, Herpes, hives, hidradenitis suppurativa, hyperhidrosis, hyperkeratosis lenticularis perstans, hypomelanotic macules, ichthyosis hystrix, impetigo, incontinentia pigmenti, infantile hemangiomas, insect bites, juvenile polyposis syndrome, Kaposi sarcoma, Kaposiform hemangioendothlioma, keloid, microcystic lymphatic malformation, keloid scar disease, keratosis follicularis dwarfism-associated skin conditions, keratosis pilaris, KID syndrome, Klippel-Trenaunay syndrome, lentigines or liver spots, Lhermitte-Duclos syndrome, lichen planopilaris, lichenoid keratosis including lichen planus, lichen sclerosus, chronic erosive oral lichen, lupus, lymphangioma circumscriptum, melanoma, Merkel cell carcinoma, metastatic melanoma, microcystic lymphatic malformation, miliaria or heat rash, Milker's nodule, Molluscum contagiosum, Muir-Torre syndrome, multiple minute digitate hyperkeratosis, myiasis including furuncular myiasis and migratory myiasis, Netherton syndrome, skin and dermal manifestations of neurofibromatosis type 1 (also referred to as “NF1” or von Recklinhausen's Disease), nevus araneus, nonmelanoma skin cancer, Olmsted syndrome, onychomycosis tinea including tinea alba, tinea pedis, tinea unguium, tinea manuum, tinea cruris, tinea corporis, tinea capitis, tinea faciei, tinea barbae, tinea imbricata, tinea nigra, tinea versicolor, tinea incognito, oral lichen planus, oral mucosal disease due to GVHD, overgrowth syndromes, pachyonychia congenita, panniculitis, paronychia, pediculosis, pemphigoid disease, pemphigus vulgaris, periungual and subungual fibroma, Peutz-Jeghers syndrome, photo-aging by UV radiation, pigmented macule, including for example nevus spilus and cafe au lait spots, pityriasis, plantar hyperkeratosis, proteus syndrome, proteus-like syndrome, pruritis vulvae, psoriasis, pyrogenic granuloma, refractory hemangioendotheliomas in Maffucci syndrome, Refsum disease, rosacea, Rosai-Dorfman disease, scabies, scleroderma, seborrheic keratosis, Sezary syndrome, Sjogren-Larsson Syndrome, squamous cell carcinoma, statis dermatitis, Sturge-Weber Syndrome, telangiectasias, trichoepithelioma, trichomoniasis, skin tumor manifestations of tuberous sclerosis, vaginal yeast infection, vascular malformations including port wine stains and lymphangiomas, vitiligo vulgaris, warts, xeroderma and xeroderma pigmentosum.

The disclosure also provides the topical rapamycin composition of any of the foregoing embodiments, for use in a method of treating a skin condition, disease, or disorder selected from the group consisting of Birt-Hogg-Dube Syndrome, cutaneous T-cell lymphoma (CTCL) dermal atrophy incident to aging or senescence, skin and dermal manifestations of neurofibromatosis type 1 (also referred to as “NF1” or von Recklinhausen's Disease), oral lichen planus, oral mucosal disease due to GVHD, pachyonychia congenita, Sturge-Weber Syndrome, vascular malformations including port wine stains and lymphangiomas.

The disclosure also provides a method for treating a skin condition, disease or disorder in a human subject in need of such treatment, the method comprising applying a topical rapamycin composition as described herein to the affected areas of the subject's skin in an amount suitable to cover the affected area with a thin layer of the composition. In embodiments, the skin condition, disease, or disorder selected from the group consisting of Acanthosis nigricans, acne, actinic keratosis, allergic conjunctivitis, ameloonychohypohidrotic syndrome, angiokeratoma, angiokeratomas in Fabry disease, angiomas including cherry angioma, senile angioma, spider angioma, strawberry angioma, and tufted angioma, athlete's foot, atopic dermatitis, bacterial vaginosis, balanitis, Bannayan-Riley-Ruvalcaba Syndrome, basal cell carcinoma, basal cell nevus Syndrome, Birt-Hogg-Dube Syndrome, blisters, blue rubber bleb nevus syndrome, bromhidrosis, Brook-Speigler Syndrome, bullous pemphigoid, calluses, candidiasis, carbunculosis, cavernous lymphangioma, cellulitis, cerebral atrophy-associated skin conditions, chelitis granulomatosis, Conradi-Eltinermann disease, Corneodermatoosseous syndrome-associated skin conditions, Cowden disease, cutaneous Castleman disease, cutaneous larva migrans, cutaneous sarcoidosis, cutaneous T-cell lymphoma (CTCL), decubitous ulcer, dermal atrophy incident to aging or senescence, dermatitis including contact dermatitis, drug-induced dermatitis, allergic dermatitis, nummular dermatitis, perioral dermatitis, neurodermatitis, seborrheic dermatitis, and atopic dermatitis, dermatofibrosarcoma protruberans, dermatophytosis, diffuse microcystic lymphatic malformations, discoid lupus erythematosus, dyshydrotic eczema, dyskeratosis congenita, ecthyma, eczema, epidermodysplasia verruciformis, epidermolysis bullosa simplex, epidermolytic ichthyosis, epithelial nevus including verrucous nevus, systematized nevus, inflammatory linear verrucous epidermal nevus, and sebaceous nevus, erysipalus, erythema multiforme, erythrokeratoderma variabilis, extramammary Paget's disease, familial cylindromatosis, familial multiple discoid fibromas, filariasis, focal acral hyperkeratosis, follicular hyperkeratosis, follicular hyperkeratosis associated with pilodental dysplasia with refractive errors, furunculosis, genital warts, gingival hypertrophy, granuloma, Hailey-Hailey disease, hemangioma simplex, hereditary footpad hyperkeratosis as afflicting dogs, Herpes, hives, hidradenitis suppurativa, hyperhidrosis, hyperkeratosis lenticularis perstans, hypomelanotic macules, ichthyosis hystrix, impetigo, incontinentia pigmenti, infantile hemangiomas, insect bites, juvenile polyposis syndrome, Kaposi sarcoma, Kaposiform hemangioendothlioma, keloid, microcystic lymphatic malformation, keloid scar disease, keratosis follicularis dwarfism-associated skin conditions, keratosis pilaris, KID syndrome, Klippel-Trenaunay syndrome, lentigines or liver spots, Lhermitte-Duclos syndrome, lichen planopilaris, lichenoid keratosis including lichen planus, lichen sclerosus, chronic erosive oral lichen, lupus, lymphangioma circumscriptum, melanoma, Merkel cell carcinoma, metastatic melanoma, microcystic lymphatic malformation, miliaria or heat rash, Milker's nodule, Molluscum contagiosum, Muir-Torre syndrome, multiple minute digitate hyperkeratosis, myiasis including furuncular myiasis and migratory myiasis, Netherton syndrome, skin and dermal manifestations of neurofibromatosis type 1 (also referred to as “NF1” or von Recklinhausen's Disease), nevus araneus, nonmelanoma skin cancer, Olmsted syndrome, onychomycosis tinea including tinea alba, tinea pedis, tinea unguium, tinea manuum, tinea cruris, tinea corporis, tinea capitis, tinea faciei, tinea barbae, tinea imbricata, tinea nigra, tinea versicolor, tinea incognito, oral lichen planus, oral mucosal disease due to GVHD, overgrowth syndromes, pachyonychia congenita, panniculitis, paronychia, pediculosis, pemphigoid disease, pemphigus vulgaris, periungual and subungual fibroma, Peutz-Jeghers syndrome, photo-aging by UV radiation, pigmented macule, including for example nevus spilus and cafe au lait spots, pityriasis, plantar hyperkeratosis, proteus syndrome, proteus-like syndrome, pruritis vulvae, psoriasis, pyrogenic granuloma, refractory hemangioendotheliomas in Maffucci syndrome, Refsum disease, rosacea, Rosai-Dorfman disease, scabies, scleroderma, seborrheic keratosis, Sezary syndrome, Sjogren-Larsson Syndrome, squamous cell carcinoma, statis dermatitis, Sturge-Weber Syndrome, telangiectasias, trichoepithelioma, trichomoniasis, skin tumor manifestations of tuberous sclerosis, vaginal yeast infection, vascular malformations including port wine stains and lymphangiomas, vitiligo vulgaris, warts, xeroderma and xeroderma pigmentosum. In embodiments, the skin condition, disease, or disorder selected from the group consisting of Birt-Hogg-Dube Syndrome, cutaneous T-cell lymphoma (CTCL) dermal atrophy incident to aging or senescence, skin and dermal manifestations of neurofibromatosis type 1 (also referred to as “NF1” or von Recklinhausen's Disease), oral lichen planus, oral mucosal disease due to GVHD, pachyonychia congenita, Sturge-Weber Syndrome, vascular malformations including port wine stains and lymphangiomas.

The disclosure also provides a method for treating facial angiofibromas in a human subject in need of such treatment, the method comprising applying a topical rapamycin composition as described herein to the affected areas of the subject's skin in an amount suitable to cover the affected area with a thin layer of the composition.

The disclosure also provides a process for making the topical rapamycin compositions described here, the process comprising preparing a solvent phase in a first container by dissolving the antioxidant in the solvent followed by adding the gel base under continuous mixing, preparing an aqueous phase in a second container by dissolving in water the surfactant, the preservative, the buffering agent, and any optional excipients, dispersing the micronized rapamycin into the aqueous phase under continuous mixing, subjecting the aqueous phase to high shear homogenization, and combining the solvent phase with the aqueous phase under continuous mixing until the solvent and aqueous phases form a homogeneous gel composition of the suspended micronized rapamycin.

Also provided are articles of manufacture or packages comprising the topical rapamycin compositions described here. In embodiments of the articles or packages, the composition is contained in a sealed or sealable epoxy coated aluminum tube.

### BRIEF DESCRIPTION OF THE DRAWINGS

FIG. 1A-C shows HPLC chromatograms using an (A) isocratic method with a rapamycin standard at 0.2 mg/ml in diluent, (B) a gradient method with rapamycin standard at 0.2 mg/ml, and (C) a gradient method using secorapamycin at 15 μg/ml. Table 1 infra provides further details of the HPLC methods.

FIG. 2 shows the results of a rapamycin pH stability screen after 1-week of storage at 5° C., 25° C. and 40° C. at pH 3, 5 and unbuffered.

FIG. 3 shows the results of solvent (10%) and temperature on rapamycin stability after 1-week of storage at 5° C., 25° C. and 40° C. PG, propylene glycol; TC, diethylene glycol monoethyl ether (Transcutol®); DMI, dimethylisosorbide.

### DETAILED DESCRIPTION

The present disclosure relates to topical rapamycin compositions and their use in methods for treating facial angiofibromas and other skin lesions associated with aberrant activation of mTOR signaling. The terms “rapamycin” and “sirolimus” may be used interchangeably to refer to the macrocyclic lactone produced by Streptomyces hygroscopicus having the molecular formula C₅₁H₇₉NO₁₃ and molecular weight 914.172 g/mol, CAS No. 53123-88-9. The disclosure provides improved topical gel formulations which exhibit both physical and chemical stability of the active ingredient, rapamycin. For example, the compositions described here demonstrate excellent physicochemical stability and are stable at ambient temperature against both rapamycin crystal growth and chemical degradation. The physicochemically stable compositions described here further provide excellent dose uniformity as well as more effective delivery of rapamycin to the target dermis layer of the skin. The formulations described here are chemically stable against the degradation of rapamycin, including its degradation into secorapamycin A or B, which is particularly disadvantageous in the context of the present topical formulations because secorapamycin may function as a poor activator of mTOR, the signaling pathway sought to be inhibited through the therapeutic application of rapamycin.

Currently, there are two commercial liquid rapamycin solutions, one oral and one injectable. The oral solution (Rapamune®) is in an aqueous base with a phospholipid excipient blend containing lecithin, glycol, sunflower oil, ascrobyl palmitate, soy fatty acids, ethanol and polysorbate 80. Storage is at 2-8° C. The injectable rapamycin solution (Torisel®; temsirolimus, a dimethylpropionic acid ester of rapamycin) is a non-aqueous solution in alcohol and propylene glycol with dl-alpha-tocopherol and citric acid. Storage is at 2-8° C. Thus, both of these commercially available rapamycin formulations require refrigeration for long-term storage and stability. In contrast, the present topical formulations provide a rapamycin gel composition that is physically and chemically stable at room temperature (25° C.) for a period of at least 6 months, preferably up to 1 year or longer.

Rapamycin (0.1%) solubilized in Transcutol® and formulated as a cream in the commercial moisturizer Vanicream™ has been used experimentally to treat angiofibromas in TSC patients (Truchuelo et al. Derm. Online J. (2012) 8:15). The present topical formulations provide physically and chemically stable rapamycin gel compositions that offer superior drug delivery to the dermis layer of the skin as compared to rapamycin formulated in Vanicream™.

These and other advantageous properties of the formulations described here provide for improved targeted delivery of rapamycin to the skin compared to reference formulations.

### Topical Rapamycin Compositions

The disclosure provides topical rapamycin formulations in the form of gel compositions comprising micronized rapamycin and methods of making same. Rapamycin is a white to off-white powder and is generally considered to be insoluble in water, having a very low solubility of only 2.6 μg/ml. Rapamycin is freely soluble in benzyl alcohol, chloroform, acetone, and acetonitrile. Isomers of rapamycin are known, e.g., isomer B and isomer C, having structures as shown in U.S. Pat. No. 7,384,953. Typically, rapamycin is a mixture of the B and C isomers. In solution, rapamycin isomers B and C interconvert and an equilibrium is achieved. It is common practice in the literature to depict the structure of rapamycin in the form of the B isomer, which is the form shown below.

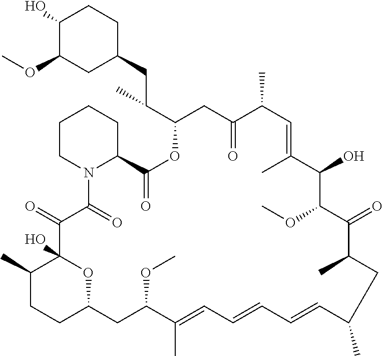 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

In embodiments of the compositions and methods described here, the API is rapamycin having an isomeric B:C ratio of less than 30:1 or less than 35:1. In embodiments, the API is rapamycin having an isomeric B:C ratio of greater than 30:1 or greater than 35:1. In one embodiment, the rapamycin has an isomer C content of 3.5% to 10%.

As a poorly water-soluble drug susceptible to non-enzymatic oxidative degradation, rapamycin presents numerous formulation challenges. In addition, and as discussed in detail in the experimental section below, rapamycin suspension formulations were found to be susceptible to the formation and growth of solid crystals, presenting additional challenges to the goal of obtaining a stable uniform suspension of rapamycin.

The particle size distribution (PSD) of the rapamycin particles in the gel compositions provided here is preferably defined by a D50 in the range of 1-5 microns. The D50 parameter refers to the size value corresponding to a cumulative size distribution at 50%, which represents the size of particles below which 50% of the sample lies. In embodiments, the rapamycin component of the formulation may further be defined by its D10 and D90 parameters, which represent the size value corresponding to cumulative size distribution at 10% or 90%. In embodiments, the particle size distribution (PSD) of the rapamycin of a gel composition described herein is defined by a D10 in the range of 0.5-1.6 microns, a D50 in the range of 1-5 microns, and a D90 in the range of 4-8 microns. PSD may be determined by methods known in the art, e.g., by laser diffraction. The PSD of the rapamycin in the topical formulations described here must be maintained within the relatively narrow ranges specified above in order to ensure content uniformity of the drug particles dispersed in the gel compositions. Lack of content uniformity, evidenced, for example, by settling or agglomeration of the drug particles, can result in inaccurate dosing and decreased bioavailability, thereby adversely affecting the safety and efficacy of the composition.

Methods for micronizing drug particles that may be used to obtain rapamycin particles having the desired PSD include jet milling, wet milling, ball milling, and high pressure homogenization.

As described in detail below in the examples, Applicants tested numerous topical compositions comprising a gel or cream structure forming base in the form of hydroxyethyl cellulose (HEC), poly(acrylic acid), and ceteareth-20/cetostearyl alcohol. The terms “poly(acrylic acid)” and “polyacrylic acid” are used interchangeably herein and “PAA” may be used herein as an abbreviation for these terms. PAA is also known by the tradename Carbomer™. Each of the gel or cream structure forming bases (which may be referred to simply as the ‘gel base’ or the ‘cream base’ herein) was formulated with each of three solvents identified in preformulation testing as lead solvents for rapamycin. These were propylene glycol (PG), diethylene glycol monoethylether (Transcutol® or TC), and dimethyl isosorbide (DMI). Preformulation work also established that rapamycin is most stable at acidic pH in the presence of an antioxidant. The preformulation studies further identified butylated hydroxyanisol (BHA) as superior to vitamin E as an antioxidant. The various formulations were subjected to tests of their physical and chemical stability over time and under different temperature conditions. This work identified four lead gel compositions as having the necessary physiochemical stability. These were subjected to further in vivo testing to determine safety (maximum tolerated dose) and efficacy (amount of rapamycin delivered to the dermal layer of the skin). As demonstrated in the example section below, the lead formulations were effective to deliver from 10 to 100 times more rapamycin to the dermis layer of the skin compared to a rapamycin formulation in Vanicream™.

Accordingly, the disclosure provides topical rapamycin gel compositions comprising a stable suspension of rapamycin in a homogeneous mixture of a gel structure forming base, a solvent, an antioxidant, a buffering agent adapted to maintain the pH of the composition at less than or equal to pH 6, and one or more optional excipients selected from a surfactant, a humectant, a chelating agent, and a preservative.

In embodiments, the pH of the composition is between 5.5 and 6.0, or the pH is between 4 and 5.

In embodiments, the micronized rapamycin is present in the gel composition in an amount of from 0.05% w/w to 2.0% w/w. In some embodiments, the rapamycin is present in the gel composition in an amount of 0.1% w/w, 0.3% w/w, or in an amount of 1.0% w/w. In embodiments, the micronized rapamycin contains less than 10% rapamycin isomer C. In embodiments, the particle size distribution (PSD) of the micronized rapamycin is defined by a D10 in the range of 1.2-1.5 microns, a D50 in the range of 2.4-2.8 microns, and a D90 in the range of 4.5-5 microns.

In embodiments, the gel composition comprises hydroxyethyl cellulose (HEC) or PAA as the gel structure forming base and a solvent selected from dimethyl isosorbide (DMI), diethylene glycol monoethylether (Transcutol or TC), and propylene glycol (PG). In an embodiment, the gel structure forming base is PAA and the solvent is selected from dimethyl isosorbide (DMI), diethylene glycol monoethylether (Transcutol or TC), and propylene glycol (PG). In an embodiment, the gel structure forming base is hydroxyethyl cellulose (HEC) and the solvent is propylene glycol. In some embodiments, the PAA is a carbomer, for example a carbomer having a molecular weight in the range of 800 to 1000, preferably 950-1000, such as Carbomer™ 980. The gel structure forming base may be present in amounts of from about 0.5 to 5% w/w, preferably from about 1-2% w/w, or 1-1.75% w/w, based on the total weight of the composition, for HEC, or about 0.1 to 3% w/w, 0.1 to 2.25% w/w, or 0.25 to 0.75% w/w, for PAA. The solvents may be present in amounts of from about 5-25% w/w, preferably 6-8% w/w, based on the total weight of the composition for DMI or TC, or about 5-25% w/w, preferably about 10-15% w/w for PG.

In embodiments, the antioxidant may be selected from alpha tocopherol, also referred to as tocopherol or vitamin E, ascorbic acid, ascorbyl palmitate, butylated hydroxyanisole, butylated hydroxytoluene, citric acid monohydrate, erythorbic acid, ethyl oleate, fumaric acid, malic acid, monothioglycerol, phosphoric acid, potassium metabisulfite, propionic acid, propyl gallate, sodium ascorbate, sodium bisulfite, sodium metabisulfite, sodium sulfite, citric acid monohydrate, tartaric acid, and thymol. In embodiments, a preferred antioxidant is butylated hydroxyanisol (BHA).

In embodiments, the composition comprises one or more optional excipients selected from a surfactant, a humectant, a chelating agent, and preservative. Generally, the optional excipients are each present in an amount of less than 2% w/w. All weight percentages in the present disclosure are based on the total weight of the gel composition.

In embodiments, the composition does not contain one or more of mineral oil, sorbitan sesquioleate, petrolatum, ceresin, methylparaben or propylparaben, PEG-6 oleate, and polyethoxylated castor oil.

In embodiments where the composition comprises a surfactant, the surfactant may be selected from a polysorbate, preferably polysorbate 80 (“PS80”, also referred to as Tween™ 80, sorbitan monooleate, or polyoxyethylene sorbitan oleate), a polyethylene glycol (PEG) fatty acid ester, including esters of lauric acid, oleic acid, and stearic acid including monoesters such as PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate; and diesters such as PEG-20 dilaurate, PEG-20 dioleate, PEG-20 distearate, PEG-32 dilaurate and PEG-32 dioleate, and mixtures of any of the foregoing mono- and di-esters. In further embodiments, the surfactant may be selected from benzalkonium chloride, benzethonium chloride, cetylpyridinium chloride, docecyl trimethyl ammonium bromide, sodium docecylsulfates, dialkyl methylbenzyl ammonium chloride, edrophonium chloride, domiphen bromide, dialkylesters of sodium sulfonsuccinic acid, sodium dioctyl sulfosuccinate, sodium cholate, and sodium taurocholate. In embodiments, the surfactant is present in an amount of less than 0.10% w/w. Preferred surfactants include polysorbate 80.

In embodiments where the composition comprises a humectant, the humectant may be selected from ammonium alginate cyclomethicone, glycerin, polydextrose, propylene glycol, sodium hyaluronate, sodium lactate, sorbitol, trehalose, triacetin, triethanolamine, and xylitol.

In embodiments where the composition comprises a chelating agent, the chelating agent may be selected from citric acid monohydrate, dipotassium edetate, disodium edetate, edetate calcium disodium, edetic acid, fumaric acid, malic acid, maltol, sodium edetate, and trisodium edetate.

In embodiments where the composition comprises a preservative, the preservative may also be an antimicrobial agent, such as an antibacterial or antifungal. A suitable preservative may be, for example, benzalkonium chloride, benzoic acid, benzyl alcohol, boric acid, bronopol, butylated hydroxyanisole, butylparaben, carbon dioxide, cetrimide, cetylpyridinium chloride, chlorbutanol, chlorhexidine, chlorobutanol, chlorocresol, chloroxylenol, cresol, dimethyl ether, ethylparaben, glycerin, hexetidine, imidurea, isopropyl alcohol, lactic acid, monothioglycerol, phenoxyethanol, phenylethyl alcohol, potassium benzoate, potassium metabisulfite, potassium sorbate, propionic acid, propyl gallate, propylene glycol, sodium acetate, sodium benzoate, sodium borate, sodium lactate, sodium metabisulfite, sodium propionate, sodium sulfite, sorbic acid, and edetic acid. In embodiments, the preservative is present in an amount of less than 1.5% w/w. A preferred preservative is benzyl alcohol.

In some embodiments, the gel composition comprises from 0.1-2.0% w/w rapamycin, or about 0.1% w/w, 0.3% w/w, 1% w/w or 2.0% w/w rapamycin, hydroxyethyl cellulose as the gel base, propylene glycol as the solvent, and butylated hydroxyanisol (BHA) as the antioxidant. In embodiments, the gel base and solvent together comprise about 15% w/w of the composition. In embodiments, the gel composition further comprises from 0.01 to 0.10% w/w polysorbate 80 and 0.5 to 1.5% w/w benzyl alcohol. In embodiments, the gel composition is physically stable against rapamycin crystal growth and chemically stable against the formation of non-enzymatic rapamycin degradation products for at least 6 months at 5° C. and 25° C. and for 3 months at 40° C. Exemplary specific embodiments of this composition are provided in Tables A-A2 below.

TABLE A
Exemplary 0.1%, 1.0%, 2.0% rapamycin gel composition.

| | | |
|---|---|---|
| | | Composition |
| Ingredient | Grade | (% w/w) |

| Citric Acid | USP | q.s. to pH 3-5 | | |
|---|---|---|---|---|
| Anhydrous | | | | |
| Sodium Citrate | USP | q.s. to pH 3-5 | | |
| Polysorbate 80 | NF | 0.025-0.25% | | |
| Butylated | USP | 0.005-0.05% | | |
| Hydroxyanisol | | | | |
| Benzyl Alcohol | USP | 0.5-3.0% | | |
| Edetate Disodium | USP | 0.01-0.25 | | |
| Propylene Glycol | USP | 5.0-25.0 | | |
| Hydroxyethyl | NF | 1.0-3.00% | | |
| Cellulose | | | | |
| (Natrasol 250 HHX) | | | | |
| Purified Water | USP | q.s. | q.s. | q.s. |
| Micronized | N/A | 0.10% | 1.00% | 2.00% |
| Rapamycin | | | | |

TABLE A1
Quantitative composition of a 0.3% rapamycin topical
gel packaged into 15 g aluminum epoxy-coated tubes.

| | | Unit | | | 15 g Tube | |
|---|---|---|---|---|---|---|
| Component | Function | (mg/g) | | Wt % | (per tube) | |
| Micronized | Active | 15.0 | g | 0.3% | 45.0 | mg |
| Rapamycin | Ingredient | | | | | |
| Citric acid | pH | 4.0 | g | 0.08% | 12.0 | mg |
| anhydrous, USP | adjustment | | | | | |
| Sodium Citrate | pH | 5.5 | g | 0.11% | 16.5 | mg |
| dihydrate, USP | adjustment | | | | | |
| Benzyl Alcohol, | Antimicro- | 50.0 | g | 1.0% | 150.0 | mg |
| NF | bial | | | | | |
| Polysorbate 80 | Surfactant | 2.5 | g | 0.05% | 7.5 | mg |
| HP, NF | | | | | | |
| Butylated | Antioxidant | 0.5 | g | 0.01% | 1.5 | mg |
| Hydroxyanisole, | | | | | | |
| NF | | | | | | |
| Edetate Disodium, | Chelating | 2.5 | g | 0.05% | 7.5 | mg |
| USP | Agent | | | | | |
| Propylene Glycol, | Solvent | 625.0 | g | 12.5% | 1.875 | mg |
| USP | | | | | | |
| Hydroxylethyl | Structure | 87.5 | g | 1.75% | 262.5 | mg |
| Cellulose, | Former | | | | | |
| (Natrosol 250 | | | | | | |
| HHX) NF | | | | | | |
| Purified Water, | Solvent | 4207.5 | g | 84.15% | 12.6 | g |
| USP | | | | | | |
| Total: | | 5000 | g | 100% | 15.0 | g |

TABLE A2
Quantitative composition of a 1.0% rapamycin topical
gel packaged into 15 g aluminum epoxy-coated tubes.

| | | Unit | | | 15 g Tube | |
|---|---|---|---|---|---|---|
| Component | Function | (mg/g) | | Wt % | (per tube) | |
| Micronized | Active | 50.0 | g | 1.0% | 150.0 | mg |
| Rapamycin | Ingredient | | | | | |
| Citric acid | pH | 4.0 | g | 0.08% | 12.0 | mg |
| anhydrous (USP) | adjustment | | | | | |
| Sodium Citrate | pH | 5.5 | g | 0.11% | 16.5 | mg |
| dihydrate (USP) | adjustment | | | | | |
| Benzyl Alcohol | Antimicro- | 50.0 | g | 1.0% | 150.0 | mg |
| (NF) | bial | | | | | |
| Polysorbate 80 | Surfactant | 2.5 | g | 0.05% | 7.5 | mg |
| HP (NF) | | | | | | |
| Butylated | Antioxidant | 0.5 | g | 0.01% | 1.5 | mg |
| Hydroxyanisole | | | | | | |
| (NF) | | | | | | |
| Edetate Disodium | Chelating | 2.5 | g | 0.05% | 7.5 | mg |
| (USP) | Agent | | | | | |
| Propylene Glycol | Solvent | 625.0 | g | 12.5% | 1.875 | mg |
| (USP) | | | | | | |
| Hydroxylethyl | Structure | 87.5 | g | 1.75% | 262.5 | mg |
| Cellulose | Former | | | | | |
| (Natrosol 250 | | | | | | |
| HHX) (NF) | | | | | | |
| Purified Water | Solvent | 4172.5 | g | 83.45% | 12.52 | g |
| (USP) | | | | | | |
| Total: | | 5000 | g | 100% | 15.0 | g |

In some embodiments, the gel composition comprises from 0.1-2.0% w/w micronized rapamycin, poly(acrylic acid) as the gel base, DMI as the solvent, and BHA as the antioxidant. In embodiments, the gel base and solvent together comprise about 8% w/w of the composition. In embodiments, the gel composition further comprises from 0.01 to 0.10% w/w polysorbate 80 and 0.5 to 1.5% w/w benzyl alcohol. In embodiments, the gel composition is physically stable against rapamycin crystal growth and chemically stable against the formation of non-enzymatic rapamycin degradation products for at least 6 months at 5° C. and for at least 2 months at 25° C. or 40° C.

In some embodiments, the gel composition comprises from 0.1-2.0% w/w micronized rapamycin, poly(acrylic acid) as the gel base, TC as the solvent, and BHA as the antioxidant. In embodiments, the gel base and solvent together comprise about 8% w/w of the composition. In embodiments, the gel composition further comprises from 0.01 to 0.10% w/w polysorbate 80 and 0.5 to 1.5% w/w benzyl alcohol. In embodiments, the gel composition is physically stable against rapamycin crystal growth and chemically stable against the formation of non-enzymatic rapamycin degradation products for at least 6 months at 5° C. and for at least 2 months at 25° C. or 40° C.

In some embodiments, the gel composition comprises 0.1-2.0% w/w micronized rapamycin, poly(acrylic acid) as the gel base, propylene glycol as the solvent, and BHA as the antioxidant. In embodiments, the gel base and solvent together comprise about 13% w/w of the composition. In embodiments, the gel composition further comprises from 0.01 to 0.10% w/w polysorbate 80 and 0.5 to 1.5% w/w benzyl alcohol. In embodiments, the gel composition is physically stable against rapamycin crystal growth and chemically stable against the formation of non-enzymatic rapamycin degradation products for at least 6 months at 5° C. and for at least 1 month at 25° C. or 40° C.

In an exemplary process for making the gel compositions described here, the gels are compounded in two phases, a solvent phase and an aqueous phase, the solvent phase containing the gel base, a solvent, and an antioxidant, the aqueous phase containing a suspension of microparticles of rapamycin dispersed in an acidified aqueous solution (pH 4-6) comprising a buffering agent, a surfactant, a preservative, and any optional excipients. In accordance with the exemplary process, the gel composition may be described as a homogeneous mixture of a solvent phase and an aqueous phase, the solvent phase comprising the gel structure forming base, or ‘gel base’, the solvent, and the antioxidant, and the aqueous phase comprising the micronized particles of rapamycin in suspension, the buffering agent, water, and the one or more optional excipients, when present.

The compositions are prepared, for example, as follows. The solvent and aqueous phases are prepared separately, and then combined. The solvent phase is prepared by dissolving the antioxidant in the solvent followed by adding the gel base under continuous mixing. The aqueous phase is prepared by dissolving, in purified water, the buffering agent, the surfactant agent, the preservative, and any optional excipients, then dispersing the micronized rapamycin into the aqueous phase under continuous mixing, followed by subjecting the aqueous phase to high shear homogenization. The solvent phase is then combined with the aqueous phase under continuous mixing until the solvent and aqueous phases form a homogeneous gel composition.

In a more detailed exemplary process, the antioxidant, BHA, is added to the solvent, propylene glycol, and mixed to dissolve. Once the BHA is completely dissolved in the solvent, the gel base, hydroxyethycellulose, is added with continuous mixing. In a separate container or vessel, a surfactant, polysorbate 80, a preservative, benzyl alcohol, a buffering agent, sodium citrate/citric acid, and a chelating agent, edatate disodium are added sequentially to purified water, allowing each ingredient to dissolve prior to adding the next ingredient. Micronized rapamycin is then added to the aqueous phase with continuous mixing until dispersed. Then, the aqueous phase is homogenized using a high shear homogenizer, e.g., a Ross High Shear Homogenizer, for 30 minutes. After homogenization, the aqueous phase is immediately subjected to continuous mixing, e.g., with a Lightnin' mixer, followed by addition of the solvent phase and continuous mixing is maintained for about 45 minutes for the gel to hydrate.

The disclosure also provides an article of manufacture or a package comprising a topical rapamycin gel composition as described herein contained in sealed or sealable epoxy-coated aluminum tubes. In embodiments, the composition is physically and chemically stable for at least 6 months at 5° C. and for at least 1, 2, 3, 5, or 6 months at 25° C. or 40° C.

### Methods of Use

The disclosure also provides compositions for the topical delivery of rapamycin to the skin, and in particular to the dermis layer of the skin, of a subject in need thereof. The disclosure also provides methods for treating a skin condition, disease, or disorder in a subject in need of such treatment by applying to the skin of the subject an effective amount of a topical rapamycin gel composition described herein. In the context of the methods described here, the terms “treat”, “treatment”, and “treating” refer to the reduction of the severity, duration, or progression of the skin lesions, for example as assessed by clinical parameters including one or more of the presence and/or degree of erythema, the average lesion size, the number or density of the lesions in an affected area, and the percent involvement. Accordingly, the efficacy of treatment may be determined, for example, by a reduction in one or more of these clinical parameters. The term “treating” may also encompass a reduction in the appearance of new skin lesions, such as facial angiofibromas, hemangiomas, vascular malformations, pyogenic granulomas, essential telangiectasias, familial multiple discoid fibromas, and cherry angiomas.

The subject in need of treatment is preferably a human subject. In embodiments, the subject is a human subject diagnosed with LAM or Tuberous Sclerosis Complex (TSC). In embodiments, the subject in need is one presenting with a skin condition, disease or disorder selected from hemangiomas, vascular malformations, pyogenic granulomas, essential telangiectasias, familial multiple discoid fibromas, and cherry angiomas. In embodiments, the vascular malformations are port wine stains or lymphangiomas. In embodiments, the subject in need is a human patient diagnosed with Proteus, Brooke-Speigler syndrome, nevus sebaceous, epidermal nevus, oral lichen planus, chelitis granulomatosis, neurofibromatosis type 1, overgrowth syndromes, or gingival hypertrophy. In the context of the present disclosure, the term “patient” generally refers to a human subject having a diagnosis.

In embodiments, the skin condition, disease, or disorder is selected from Acanthosis nigricans, acne, actinic keratosis, allergic conjunctivitis, ameloonychohypohidrotic syndrome, angiokeratoma, angiokeratomas in Fabry disease, angiomas including cherry angioma, senile angioma, spider angioma, strawberry angioma, and tufted angioma, athlete's foot, atopic dermatitis, bacterial vaginosis, balanitis, Bannayan-Riley-Ruvalcaba Syndrome, basal cell carcinoma, basal cell nevus Syndrome, Birt-Hogg-Dube Syndrome, blisters, blue rubber bleb nevus syndrome, bromhidrosis, Brook-Speigler Syndrome, bullous pemphigoid, calluses, candidiasis, carbunculosis, cavernous lymphangioma, cellulitis, cerebral atrophy-associated skin conditions, chelitis granulomatosis, Conradi-Eltinermann disease, Corneodermatoosseous syndrome-associated skin conditions, Cowden disease, cutaneous Castleman disease, cutaneous larva migrans, cutaneous sarcoidosis, cutaneous T-cell lymphoma (CTCL), decubitous ulcer, dermal atrophy incident to aging or senescence, dermatitis including contact dermatitis, drug-induced dermatitis, allergic dermatitis, nummular dermatitis, perioral dermatitis, neurodermatitis, seborrheic dermatitis, and atopic dermatitis, dermatofibrosarcoma protruberans, dermatophytosis, diffuse microcystic lymphatic malformations, discoid lupus erythematosus, dyshydrotic eczema, dyskeratosis congenita, ecthyma, eczema, epidermodysplasia verruciformis, epidermolysis bullosa simplex, epidermolytic ichthyosis, epithelial nevus including verrucous nevus, systematized nevus, inflammatory linear verrucous epidermal nevus, and sebaceous nevus, erysipalus, erythema multiforme, erythrokeratoderma variabilis, extramammary Paget's disease, familial cylindromatosis, familial multiple discoid fibromas, filariasis, focal acral hyperkeratosis, follicular hyperkeratosis, follicular hyperkeratosis associated with pilodental dysplasia with refractive errors, furunculosis, genital warts, gingival hypertrophy, granuloma, Hailey-Hailey disease, hemangioma simplex, hereditary footpad hyperkeratosis as afflicting dogs, Herpes, hives, hidradenitis suppurativa, hyperhidrosis, hyperkeratosis lenticularis perstans, hypomelanotic macules, ichthyosis hystrix, impetigo, incontinentia pigmenti, infantile hemangiomas, insect bites, juvenile polyposis syndrome, Kaposi sarcoma, Kaposiform hemangioendothlioma, keloid, microcystic lymphatic malformation, keloid scar disease, keratosis follicularis dwarfism-associated skin conditions, keratosis pilaris, KID syndrome, Klippel-Trenaunay syndrome, lentigines or liver spots, Lhermitte-Duclos syndrome, lichen planopilaris, lichenoid keratosis including lichen planus, lichen sclerosus, chronic erosive oral lichen, lupus, lymphangioma circumscriptum, melanoma, Merkel cell carcinoma, metastatic melanoma, microcystic lymphatic malformation, miliaria or heat rash, Milker's nodule, Molluscum contagiosum, Muir-Torre syndrome, multiple minute digitate hyperkeratosis, myiasis including furuncular myiasis and migratory myiasis, Netherton syndrome, skin and dermal manifestations of neurofibromatosis type 1 (also referred to as “NF1” or von Recklinhausen's Disease), nevus araneus, nonmelanoma skin cancer, Olmsted syndrome, onychomycosis tinea including tinea alba, tinea pedis, tinea unguium, tinea manuum, tinea cruris, tinea corporis, tinea capitis, tinea faciei, tinea barbae, tinea imbricata, tinea nigra, tinea versicolor, tinea incognito, oral lichen planus, oral mucosal disease due to GVHD, overgrowth syndromes, pachyonychia congenita, panniculitis, paronychia, pediculosis, pemphigoid disease, pemphigus vulgaris, periungual and subungual fibroma, Peutz-Jeghers syndrome, photo-aging by UV radiation, pigmented macule, including for example nevus spilus and cafe au lait spots, pityriasis, plantar hyperkeratosis, proteus syndrome, proteus-like syndrome, pruritis vulvae, psoriasis, pyrogenic granuloma, refractory hemangioendotheliomas in Maffucci syndrome, Refsum disease, rosacea, Rosai-Dorfman disease, scabies, scleroderma, seborrheic keratosis, Sezary syndrome, Sjogren-Larsson Syndrome, squamous cell carcinoma, statis dermatitis, Sturge-Weber Syndrome, telangiectasias, trichoepithelioma, trichomoniasis, skin tumor manifestations of tuberous sclerosis, vaginal yeast infection, vascular malformations including port wine stains and lymphangiomas, vitiligo vulgaris, warts, xeroderma and xeroderma pigmentosum.

In embodiments, the skin condition, disease, or disorder is selected from Birt-Hogg-Dube Syndrome, cutaneous T-cell lymphoma (CTCL) dermal atrophy incident to aging or senescence, skin and dermal manifestations of neurofibromatosis type 1 (also referred to as “NF1” or von Recklinhausen's Disease), oral lichen planus, oral mucosal disease due to GVHD, pachyonychia congenita, Sturge-Weber Syndrome, vascular malformations including port wine stains and lymphangiomas.

The topical rapamycin compositions described here are effective to deliver a therapeutically effective amount of rapamycin to the dermis layer of the skin of a subject in need thereof. In embodiments, a composition described here applied as a 20 mg dose is effective to deliver from 16-41 μg rapamycin to 1 g of dermis layer of the skin in a minipig assay.

The compositions described here are particularly useful in methods for delivering a therapeutically effective amount of rapamycin to the skin of a subject while avoiding systemic exposure. Preferably, a subject administered rapamycin via a topical composition as described here will exhibit blood levels of rapamycin of less than 1 or 2 ng/ml within 12 to 24 hours after application of the composition to the skin of the subject.

In embodiments, the amount of rapamycin in a composition described here is an amount effective to treat facial angiofibromas, or other skin lesions, including hemangiomas, vascular malformations, pyogenic granulomas, essential telangiectasias, familial multiple discoid fibromas, and cherry angiomas.

In embodiments, the effective amount of rapamycin is the amount applied to the skin according to the methods described here for application of a topical rapamycin composition. For example, in accordance with the methods described here, the amount of the composition applied to the affected area is generally in the range of about 5 cubic centimeters (cm³), or from about 5-20 cm³, or about 15-20 cm³. In accordance with the methods described here, the composition is applied to the affected area of skin, which is the area of skin comprising the lesions to be treated, in an amount suitable to cover the affected area with a thin layer of the composition, for example an amount in the range of about 5-20 cm³, or from about 15-20 cm³ applied to the affected area, preferably once daily or twice daily. In embodiments, the application is once daily. In embodiments, the topical rapamycin composition is applied topically to affected regions, such as the face, of a patient. In embodiments, a pump is used to dispense a defined amount of the composition, for example about 1 gm (or about 5-20 cm³ or from about 15-20 cm³). The dispensed amount is applied to affected regions of the skin and allowed to remain, preferably overnight, without wetting or washing. In embodiments, the composition is applied once daily. In embodiments, the composition is applied twice or three times daily. The composition is preferably stored and used at room temperature.

Further embodiments will become apparent from the following examples which illustrate the invention in some of its major aspects but is not intended to limit the scope in any way thereof.

### EXAMPLES

The following sections describe the formulation of topical rapamycin compositions that meet the needs discussed above. The examples describe the unique challenges of formulating rapamycin as the active pharmaceutic ingredient (API) in a semi-solid composition, including the difficulties of obtaining a homogeneous suspension of the API in the composition, protecting the API against chemical degradation over time in storage and at increased temperatures, and avoiding crystal growth of the API in the formulation. The examples further provide solutions to these problems in the form of the chemically and physically stable compositions described herein.

Preformulation work was undertaken to establish a suitable solvent system. Example 1 describes the solvents tested and the selection of three lead solvents for further formulation. These initial studies also established that the composition would take the form of a stable homogeneous suspension of rapamycin. Examples 2 and 3 describe the experimentation undertaken to arrive at a composition having the required stability.

Preformulation work also involved development of a reliable, robust HPLC method for detecting rapamycin and any degradation products, pH stability testing, topical solvent screening, solvent compatibility testing and antioxidant screening. The preformulation stage was followed by prototype rapamycin formulation development. This consisted of the preparation of 8-10 cream and gel vehicles containing rapamycin for stability testing (based on appearance and viscosity) after 1-month at 40° C. Compounding was tested on 6-8 active formulations initially testing for appearance, viscosity, and pH in aqueous vehicle bases. Downstream testing on the most desirable preformulations was performed at 1-month and 3-months storage at 25° C. and either 5° C. or 40° C. depending on preformulation data. These experiments led to four lead formulations and are described below in Examples 1-3.

Lead formulations were tested in a preclinical minipig model for tolerability and delivery of rapamycin to the skin. In summary, these experiments demonstrated that all four lead formulations were effective at delivering higher amounts of rapamycin to the skin compared with a Vanicream™ formulation. These experiments are described below in Example 4.

### Example 1: Preformulation

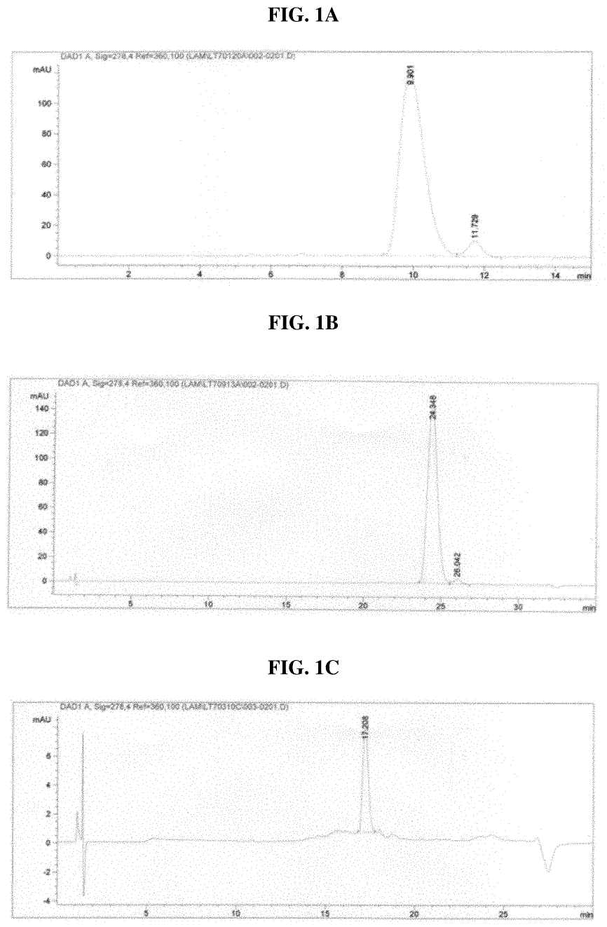

Rapamycin was detected by high pressure liquid chromatography analysis (HPLC) using a modified isocratic method in order to efficiently detect secorapamycin, a possible impurity/degradation product of rapamycin. HPLC analysis was performed with an Agilent 1200 instrument using a UV-visible detector. Specifications of two HPLC methods are shown in Table 1. The first method, designated RAP_1_LC.M, was linear over 0.05 to 0.4 mg/ml with a correlation coefficient >0.999 and % relative standard deviation (RSD) on repeat injections of <2%, making it sufficient for preformulation work. FIG. 1A shows an HPLC chromatogram of the rapamycin standard with a possible impurity/degradation product, secorapamycin, eluting within or very close to the diluent front. In order to more accurately detect secorapamycin, an acetonitrile gradient was used from 10% to 55% and the gradient time was from 2.5 to 15 minutes; the method run time was also increased to 35 minutes. In addition, and based on results from pH-stability screening (discussed below), the acetate buffer was replaced with a pH 4.5 20 mM phosphate buffer. Since the method run time was longer at 55° C., the buffer pH was reduced to minimize potential for degradation during the assay. This method is designated RAP_2_LC.M in Table 1. The RAP_2_LC.M gradient method was linear over a range of 0.05 to 0.4 mg/ml with a correlation coefficient of >0.999. The % RSD on repeat injections was <2%. A chromatogram is shown in FIG. 1B with 0.2 mg/ml rapamycin, along with a chromatogram of only pure 15 μg/mL secorapamycin (FIG. 1C). Secorapamycin eluted at a Relative Retention Time (RRT) of ˜0.7 with respect to the main rapamycin peak (FIG. 1A-B). Based on these results, the RAP_2_LC.M gradient method was considered to be acceptable for rapamycin analysis in prototype formulations development and testing.

TABLE 1
HPLC methods used for preformulation and formulation analysis.

| | Preformulation | Formulation |
|---|---|---|
| | Method | Method |
| | RAP_1_LC.M | RAP_2_LC.M |
| Column | Phenomenex | Phenomenex |
| | Hyperclone, 3 | Hyperclone, 3 |
| | μm BDS C18, | μm BDS C18, |
| | 150 × 4.6 mm | 150 × 4.6 mm |
| Sample | Ambient | Ambient |
| Temperature | | |
| Column | 55° C. | 55° C. |
| Temperature | | |
| Injection | 25 μL | 25 μL |
| Volume | | |
| Flow Rate | 1.5 mL/min | 1.25 mL/min |
| Detection | 278 nm | 278 nm |
| Mobile Phase | Buffer: 10 mM | Buffer: 20 mM |
| | ammonium acetate, | sodium phosphate, |
| | pH 5.8 | pH 4.5 |
| | A: 58% | A: 10% |
| | Acetonitrile/ | Acetonitrile/ |
| | 42% buffer | 90% buffer |
| | | B: Acetonitrile |
| Gradient | Isocratic | 0 min: 0% B |
| | | 2.5 min: 0% B |
| | | 15 min: 55% B |
| | | 30 min: 55% B |
| | | 30.1 min: 0% B |
| Run time | 15 min | 35 min |
| Diluent for | 50% ACN/water | 50% ACN/water |
| standards | with 0.05% w/v | with 0.05% w/v |
| and samples | citric acid | citric acid |
| | monohydrate | monohydrate |
| | (0.5 g/L) | (0.5 g/L) |
| Standard | 0.2 mg/mL | 0.2 mg/mL |
| | rapamycin in | rapamycin in |
| | diluent | diluent |
| | (diluted if | |
| | needed to | |
| | assay lower | |
| | concentrations) | |

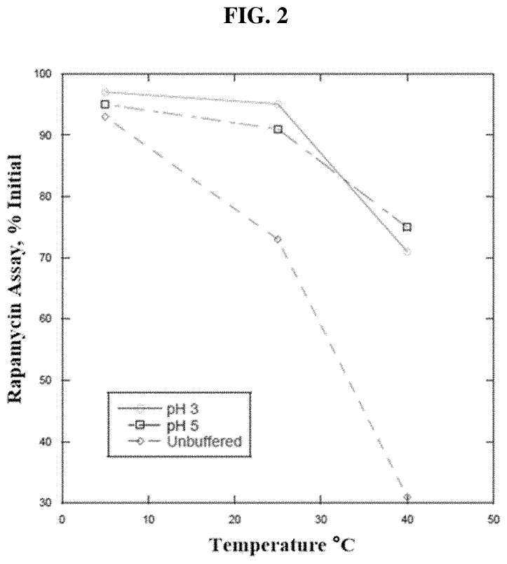

Next, we tested the pH stability of rapamycin using a 1:1 ethanol:water blend in which rapamycin has a solubility >0.1 mg/ml. 20 mM citrate buffers were prepared at pH 3, 5 and 7. With the exception of the pH 7 buffer, these buffers were miscible in 50% ethanol. Rather than reduce the buffer concentration to obtain a higher pH, the third solution was prepared with unbuffered water. An Orion 710A+ pH meter with a Thermo Scientific electrode was used for pH measurement. Solutions of rapamycin at 0.1 mg/mL and at pH 3, 5 and 7 were prepared and stored at 5° C., 25° C. and 40° C. for 1 week. The results, shown in FIG. 2, demonstrate that rapamycin is more stable at an acidic pH.

We next tested the solubility of rapamycin in ten different solvents, as shown in Table 2. Solubility screening was performed by gravimetric analysis. In a glass vial, known amounts of API were added to a known amount of solvent and the sample was rotated with a vial rotator (VWR) at room temperature. If the API completely dissolved, an additional known amount was added. The samples were rotated for at least 48 hours after the last API addition before determining the gravimetric result (i.e., greater than a % w/w or less than a % w/w). These tests indicated that rapamycin was most highly soluble in dimethyl isosorbide (DMI) and diethylene glycol monoethyl ether (TC).

TABLE 2
Solubility of rapamycin in various solvents by gravimetric analysis.

| | Rapamycin |
|---|---|
| Solvent | solubility % w/w |
| Propylene glycol (PG) | <0.75 |
| PEG 400 | <1.5 |
| Diethylene glycol monoethyl | >13 |
| ether (TC) | |
| Dimethyl isosorbide (DMI) | >7 |
| Light mineral oil | <0.3 |
| Medium chain triglycerides | <0.1 |
| Diisopropyl adipate | <1.0 |
| Hexylene glycol | <1.0 |
| Octyldodecanol | <1.0 |
| Isopropyl myristate | <0.1 |

Rapamycin's saturation solubility in TC and DMI was 15.5% and 20.6% w/w, respectively. To estimate the effect of solvents on the aqueous solubility of rapamycin, the saturation solubility was measured in water and 10% solutions of selected solvents. All solutions were buffered with 5 mM citrate buffer at pH 4.5. PG had a negligible effect on solubility while TC and DMI increased solubility by ˜1.5-2.0 μg/mL. The results are summarized in Table 3.

TABLE 3
Solubility of rapamycin in a 10% co-solvent aqueous solution
with various solvents buffered with 5 mM citrate.

| Solvent (buffered at pH | Rapamycin |
|---|---|
| 4.5, 5 mM Citrate) | Solubility, mcg/mL |
| Water | 2.9 |
| 10% Propylene Glycol (PG) | 2.9 |
| 10% Diethylene Glycol | 4.6 |
| Monoethyl Ether (TC) | |
| 10% Dimethyl Isosorbide | 5.2 |
| (DMI) | |

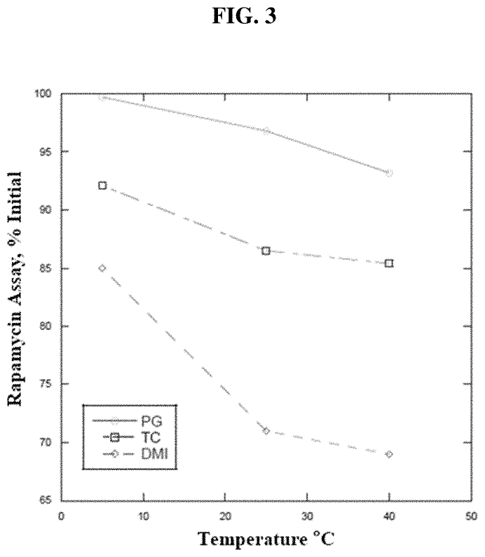

We next tested the stability of rapamycin in three different solvents, propylene glycol (PG), diethylene glycol monoethyl ether (TC), and dimethyl isosorbide (DMI), as shown in FIG. 3. Solutions of rapamycin (0.2% w/w) were prepared in each of the three solvents, DMI, TC, and PG, and stored at 5° C., 25° C., and 40° C. for 1 week. FIG. 3 shows that rapamycin was most compatible with propylene glycol (PG) and least compatible with dimethyl isosorbide (DMI).

We next performed an antioxidant screen to identify antioxidants that stabilized rapamycin. Since there was significant degradation in diethylene glycol monoethyl ether (TC) after 1 week, and TC can generate peroxides, it was selected as the solvent for the antioxidant screen. Rapamycin solutions (0.2% w/w) were prepared in Transcutol® with the following antioxidants: (1) No antioxidant; (2) 0.01% butylated hydroxyanisol (BHA); (3) 0.002% dl-alpha-tocopherol (vitamin E); and (4) 0.01% BHA+0.002% vitamin E. The results after 1-, 2-, and 4-weeks of storage are shown in Table 4.

TABLE 4
Antioxidant effect on rapamycin solution
stability at different temperatures.

| | | Rapamycin Assay (% of Initial) | | | |
|---|---|---|---|---|---|
| Temp | | No | | | BHA + |
| ° C. | Week | Antioxidant | BHA | Vit. E | Vit. E |
| 25 | 1 | 95.7 | 99.2 | 98.3 | 98.8 |
| | 2 | Not done | Not done | Not done | Not done |
| | 4 | 84.6 | 98.3 | 98.2 | 98.7 |
| 40 | 1 | 87.3 | 97.1 | 96.8 | 96.9 |
| | 2 | 79.0 | 98.1 | 97.0 | 98.7 |
| | 4 | 60.8 | 94.4 | 82.2 | 96.2 |

Incorporation of an antioxidant significantly improved solution stability at both temperatures and at all time points. The 4-week data at 40° C. was used to calculate the main effects of the two antioxidants and their interaction (Box et al., 1978) for the assay values. The positive effect of BHA (+23.8) was twice the positive effect of Vit. E (+11.6). The interaction has a negative value (−9.8) since vitamin E is a weaker antioxidant and, when vitamin E was combined with BHA, it did not significantly improve the assay value when BHA was used alone. Based on these results, and its water solubility, BHA was selected as the antioxidant for rapamycin formulations.

In summary, the main results from the preformulation work showed that: (1) rapamycin is most stable at acidic pH; three promising solvents were identified, propylene glycol (PG), diethylene glycol monoethylether (Transcutol® or TC), and dimethyl isosorbide (DMI); BHA was shown to be a suitable antioxidant for maintaining rapamycin stability; and based on solubility and solution stability, an aqueous-based formulation, suitable for facial application, would need to be a suspension formulation.

### Example 2: Cream and Gel Vehicle Formulations

A series of 1 cream and 2 gels were prepared. Gels of series 1 (Gel 1A, 1B, and 1C) utilized hydroxyethyl cellulose (HEC) and gels of series 2 (Gel 2A, 2B, and 2C) utilized Carbomer 980 as the structure former. The cream series (Cream A, Cream B, Cream C) utilized Ceteareth-20 and Cetostearyl alcohol, as detailed below in Table 5. The other primary difference among the tested formulations was the solvent. Gels and Creams designated “A” utilized dimethyl isosorbide (DMI), while those designated “B” utilized diethylene glycol monoethylether (Transcutol® or TC), and those designated “C” utilized propylene glycol (PG).

TABLE 5
Cream and gel base vehicles screened
for structure-forming ingredients.

| Vehicle | Structure forming | |
|---|---|---|
| base | ingredient(s) | Rationale |
| Gel 1 | Hydroxyethyl | Maximum approved level, |
| | cellulose or | can form a gel at pH |
| | HEC*: 1.75% w/w | 4.5 (most stable for |
| | | rapamycin) |
| Gel 2 | Carbomer 980: | Firm gel at pH 5.5-6.0, |
| | 0.5% w/w | below maximum approved |
| | | level |
| Cream | Ceteareth-20**: | Capable of emulsifying |
| | 1.25% w/w | emollients without a |
| | Cetostearyl | “soapy” skin-feel, |
| | alcohol: 6.5% | smooth/spreadable cream |
| | | at pH 4.5 (most stable |
| | | for rapamycin), below |
| | | maximum approved levels |

*Natrosol 250 HXX (Ashland)
**Polyoxyl 20 cetostearyl ether.
Each vehicle base was formulated with one of the three solvents identified in the preformulation work: “A” for 7.5% w/w DMI, “B” for 7.5% w/w TC, and “C” for 12.5% w/w PG. The vehicle compositions are summarized in the table below where many ingredients are color-coded for their functions. Compounding techniques will be described for the active formulations later in the document.

Rapamycin stability in each of the nine formulation was tested initially at 40° C. for 1 month for changes in appearance and viscosity. Viscosity measurements were performed with a Brookfield rotational viscometer. The parameters for each formulation type were: hydroxyethylcellulose gels: (1) RV viscometer, Spindle #14, 12 rpm; (2) carbomer gels: LV viscometer, helipath stand, spindle #95, 3 rpm; and (3) Cream: LV viscometer, helipath stand, spindle #95, 0.3 rpm. As shown in Table 6 and Table 7, there were no significant changes in pH, viscosity, or appearance.

Each of the nine formulations was also subjected to 3 freeze/thaw cycles: 3 days at −20° C. followed by 4 days at room temperature and evaluated for appearance after each cycle. No changes in appearance were observed. Based on these results, all nine vehicles would be suitable for compounding with micronized rapamycin.

TABLE 6
Initial rapamycin vehicle test results at 40° C.

| | | Viscosity, | |
|---|---|---|---|
| Formulation | pH | cP | Appearance |
| Gel 1A | 4.55 | 23,900 | Clear to translucent |
| | | | soft gel |
| Gel 1B | 4.47 | 24,800 | Clear to translucent |
| | | | soft gel |
| Gel 1C | 4.46 | 24,300 | Clear to translucent |
| | | | soft gel |
| Gel 2A | 5.62 | 85,200 | Clear firm gel |
| Gel 2B | 5.83 | 93,500 | Clear firm gel |
| Gel 2C | 5.85 | 115,000 | Clear firm gel |
| Cream A | 4.45 | 530,000 | White to off-white |
| | | | homogeneous cream |
| Cream B | 4.52 | 504,000 | White to off-white |
| | | | homogeneous cream |
| Cream C | 4.51 | 511,000 | White to off-white |
| | | | homogeneous cream |

TABLE 7
Rapamycin vehicle stability testing results after 1 month at 40° C.

| | | Viscosity, | |
|---|---|---|---|
| Formulation | pH | cP | Appearance |
| Gel 1A | 4.41 | 23,200 | Clear to translucent |
| | | | soft gel |
| Gel 1B | 4.65 | 24,600 | Clear to translucent |
| | | | soft gel |
| Gel 1C | 4.56 | 24,100 | Clear to translucent |
| | | | soft gel |
| Gel 2A | 5.66 | 84,600 | Clear firm gel |
| Gel 2B | 5.77 | 93,600 | Clear firm gel |
| Gel 2C | 5.79 | 112,000 | Clear firm gel |
| Cream A | 4.39 | 556,000 | White to off-white |
| | | | homogeneous cream |
| Cream B | 4.65 | 517,000 | White to off-white |
| | | | homogeneous cream |
| Cream C | 4.64 | 519,000 | White to off-white |
| | | | homogeneous cream |

### Example 3: Compounding of Rapamycin and Stability Testing

The following is a summary of the compounding steps for rapamycin formulations on the 100-300 g scale. During compounding, the API dispersion uniformity was checked by examining a small sample microscopically. For API compounding, low-shear mixing was performed with a stainless steel propeller blade (1.5″ diameter) and an IKA Eurostar 200 overhead mixer. High-shear mixing was performed with a GLH homogenizer using a 10 mm stainless steel rotor-stator head.

### Gel 1: Hydroxyethyl Cellulose (HEC) Base

Formation of the hydroxyethyl cellulose gel base requires the following preparative steps: in the main vessel, adding water (reserve 5% for rinsing), citrates (acid and salt), EDTA, glycerin (if used), polysorbate 80, and benzyl alcohol; mixing with a propeller blade on an overhead mixer until homogeneous; adding API; mixing until the solids are dispersed (˜10-20 min); starting high-shear mixing using a 10 mm rotor/stator homogenizer, continuing homogenization for at least 20 minutes and returning to propeller mixing after homogenization; in a separate slurry vessel, combining the solvent (dimethyl isosorbide, diethylene glycol monoethyl ether, or propylene glycol), BHA, phenoxyethanol (if using) and HEC and mixing until the polymer slurry is uniform and smooth; adding slurry vessel contents to the main vessel with propeller blade mixing and rinsing slurry vessel residual contents into the main vessel with reserved rinse water; increasing mixing speed to keep the gel moving while the HEC hydrates; and mixing the formulation for at least 60 min. to allow hydration of the HEC using a stainless steel spatula for side scraping, if needed.

### Gel 2: Carbomer 980 Base

Formation of the Carbomer 980 gel base requires the following preparative steps: in the main vessel, adding water (reserve 5% for rinsing), EDTA, glycerin (if used), polysorbate 80, and benzyl alcohol; mixing with a propeller blade on an overhead mixer until homogeneous; adding API; mixing until the solids are dispersed (˜10-20 min); starting high-shear mixing using a 10 mm rotor/stator homogenizer, continuing homogenization for at least 20 minutes and returning to propeller mixing after homogenization; in a separate solvent vessel, combining the solvent (dimethyl isosorbide, diethylene glycol monoethyl ether, or propylene glycol), BHA, and phenoxyethanol (if using) and mixing until the solvent phase is uniform; adding solvent vessel's contents to the main vessel with propeller blade mixing, rinsing solvent vessel residual into main vessel with reserved water and mixing the formulation for at least 10 min.; slowly adding the carbomer 980 powder to the main vessel and mixing for at least 45 min. after addition is complete; and adding sodium hydroxide solution and increase propeller speed as the gel thickens using a stainless steel spatula for side scraping, if needed. pH of the gel should be 5.5-6.0; adjusting pH as needed with additional mixing.

### Cream Base Vehicles

Formation of the Carbomer 980 gel base requires the following preparative steps: in the main vessel, add water, EDTA, glycerin (if used), citrates (acid and salt), and 10% of the ceteareth-20; mixing with a propeller blade on an overhead mixer until homogeneous; adding API and mixing until the solids are dispersed (˜10-20 min); starting high-shear mixing using a 10 mm rotor/stator homogenizer, continuing homogenization for at least 20 minutes and returning to propeller mixing after homogenization heating the main vessel contents to 60-65° C.; in a separate lipids vessel, combining the emollients, cetostearyl alcohol, remaining ceteareth-20, BHA, and parabens and heating to 60-65° C. and mixing until the lipids vessel contents are uniform to maintain a temperature at 60-65° C.; adding lipids vessel's contents to the main vessel with propeller blade mixing; mixing the formulation for at least 10 min. and starting high-shear mixing using a 10 mm rotor/stator homogenizer; cooling the main vessel while continuing high-shear mixing; when the main vessel contents reach 45° C., stopping high-shear mixing and returning to propeller mixing; and continuing mixing, using a stainless steel spatula for side scraping as needed, until it reaches 30° C.

For prototype active formulations, the rapamycin concentration was 1.0% w/w, or 10 mg/ml. Based on experience with the rapamycin method and preformulation work, the following practices were used for extraction: 0.5 g of formulation would be added to a 25 mL flask (0.2 mg/mL rapamycin); 50% acetonitrile with 0.05% citric acid (HPLC diluent) would be used to ensure solubility and stability in the diluent; and gentle heating would be used to disperse the cream formulation. One vehicle from each formulation base (Gel 1, Gel 2, or Cream) was selected since the primary difference within each formulation base was the solvent. The “B” formulations were selected for the extraction confirmation since they contained Transcutol®, which solubilized rapamycin at an intermediate level in the three solvents. Gel extraction was tested using the following steps of adding 0.5 g of Gel 1B or Gel 2B vehicle to a volumetric flask; adding 1 mL of 5 mg/ml rapamycin in acetonitrile, adding 15 mL of HPLC diluent to the vial and vortexing to disperse/dissolve the gel; adding a small stir bar and mixing for 15 minutes; removing stir bar, bringing flask to volume with HPLC diluent and mixing; and filtering an aliquot through a 0.45 micron nylon syringe filter into an amber HPLC vial for analysis.

Cream extraction was tested according to the following procedure: adding 0.5 g of Cream B vehicle to a volumetric flask; adding 1 ml of 5 mg/ml rapamycin in acetonitrile, adding 15 mL of HPLC diluent to the vial and vortex to disperse the cream; placing the flask in a 50° C. water bath and gently swirling to periodically melt/disperse cream; removing from bath, adding a small stir bar and mixing for 15 minutes; removing stir bar and adding HPLC diluent to slightly below the fill line and allowing the flask to stand for 20 minutes for temperature equilibration; and bringing the flask up to volume with HPLC diluent, mixing, and filtering an aliquot through a 0.45 micron nylon syringe filter into an amber HPLC vial for analysis. Extraction experiments were performed in duplicate and the results are shown in Table 8.

TABLE 8
Extraction and recovery of rapamycin
formulations in different vehicles.

| | % recovery, | % recovery, |
|---|---|---|
| Vehicle | Extraction 1 | Extraction 2 |
| Gel 1B | 99.3 | 100.1 |
| Gel 2B | 100.4 | 100.3 |
| Cream B | 100.6 | 99.2 |

All of the recovery results were between 99 and 101%. Based on these results, the extraction methods were considered to be suitable for rapamycin active formulations.

Seven formulations were selected for compounding with 1% micronized rapamycin: Gel 1A; Gel 1B; Gel 1C; Gel 2A; Gel 2B; Gel 2C; and Cream C. A decision was made to focus on gel formulations, in part, due to agglomeration in the creams. Cream B was kept to provide comparative data. Table 9 shows the initial results obtained from compounding each of the 7 formulations with rapamycin and testing for uniformity of drug content in the top, middle, and bottom layers of the formulation using the extraction methods discussed above.

TABLE 9
Initial testing of the 7-prototype 1.0% rapamycin formulations.

| | Assay | | Viscosity | |
|---|---|---|---|---|
| Formulation | % w/w¹ | pH | cP | Appearance |
| Gel 1A | 1.02 | 4.65 | 24,700 | Conforms |
| | 0.99 | | | (white to off- |
| | 1.01 | | | white soft gel) |
| Gel 1B | 1.00 | 4.68 | 26,400 | Conforms |
| | 0.98 | | | (white to off- |
| | 1.01 | | | white soft gel) |
| Gel 1C | 1.01 | 4.68 | 25,300 | Conforms |
| | 1.01 | | | (white to off- |
| | 1.00 | | | white soft gel) |
| Gel 2A | 1.01 | 5.89 | 121,000 | Conforms |
| | 0.97 | | | (White to off- |
| | 1.01 | | | white firm gel) |
| Gel 2B | 0.98 | 5.67 | 121,000 | Conforms |
| | 1.02 | | | (White to off- |
| | 1.00 | | | white firm gel) |
| Gel 2C | 1.02 | 5.78 | 138,000 | Conforms |
| | 0.99 | | | (White to off- |
| | 0.99 | | | white firm gel) |
| Cream C | 0.96 | 4.70 | 586,000 | Conforms |
| | 0.91 | | | (White to off-white |
| | 1.08 | | | homogeneous cream) |

¹Listed in order from Top, Middle, Bottom

Each of the gel formulations demonstrated homogeneous distribution of rapamycin throughout the composition and no significant change in the pH or viscosity of the respective vehicles. In contrast, the content uniformity for the cream was poor (% RSD of 9.0). Microscopic examination of Cream C showed large agglomerates of fine crystalline particles in the formulation. The microscopic quality of rapamycin particle dispersion was checked through the process. For Cream C, the rapamycin dispersion only showed evidence of agglomeration after the oil phase was added, even while mixing with high shear.

Each of the six formulations were next tested for stability of rapamycin following 1-month storage at either 25° C. or 40° C. (Table 10). There was no significant change in the content uniformity of the rapamycin, the vehicle pH or viscosity, or in the bulk appearance for any of the gel formulations. No new impurity peaks were identified, including any secorapamycin formation.

While there was no significant change in content uniformity for Cream C, two results were outside 90-110% of the target concentration. This is characteristic of a formulation with low content uniformity due to the agglomeration of micronized rapamycin particles. Evidence of rapamycin crystal growth was also observed in Cream C.

TABLE 10
One-month stability results for the 7 prototype 1.0% rapamycin formulations.

| | | Assay | | Viscosity | |
|---|---|---|---|---|---|
| Formulation | Temp. | % w/w¹ | pH | cP | Appearance |
| Gel 1A | 25° C. | 1.03 | 4.69 | 24,900 | Conforms - white to |
| | | 1.02 | | | off-white soft gel |
| | | | | | Evidence of Crystal |
| | | | | | Growth |
| | 40° C. | 1.02 | 4.73 | 25,900 | Conforms - white to |
| | | 1.02 | | | off-white soft gel |
| | | | | | Evidence of Crystal |
| | | | | | Growth |
| Gel 1B | 25° C. | 1.02 | 4.65 | 26,100 | Conforms - white to |
| | | 1.02 | | | off-white soft gel |
| | | | | | Evidence of Crystal |
| | | | | | Growth |
| | 40° C. | 1.02 | 4.66 | 25,600 | Conforms - white to |
| | | 1.03 | | | off-white soft gel |
| | | | | | Evidence of Crystal |
| | | | | | Growth |
| Gel 1C | 25° C. | 0.98 | 4.72 | 25,500 | Conforms - white to |
| | | 1.00 | | | off-white soft gel |
| | | | | | No Evidence of |
| | | | | | Crystal Growth |
| | 40° C. | 1.01 | 4.68 | 26,000 | Conforms - white to |
| | | 1.00 | | | off-white soft gel |
| | | | | | No Evidence of |
| | | | | | Crystal Growth |
| Gel 2A | 25° C. | 1.02 | 5.82 | 121,900 | Conforms - white to |
| | | 1.02 | | | off-white firm gel |
| | | | | | No Evidence of |
| | | | | | Crystal Growth |
| | 40° C. | 1.00 | 5.72 | 122,900 | Conforms - white to |
| | | 1.02 | | | off-white firm gel |
| | | | | | No Evidence of |
| | | | | | Crystal Growth |
| Gel 2B | 25° C. | 1.02 | 5.70 | 125,300 | Conforms - white to |
| | | 1.01 | | | off-white firm gel |
| | | | | | No Evidence of |
| | | | | | Crystal Growth |
| | 40° C. | 1.02 | 5.83 | 124,400 | Conforms - white to |
| | | 0.99 | | | off-white firm gel |
| | | | | | No Evidence of |
| | | | | | Crystal Growth |
| Gel 2C | 25° C. | 1.02 | 5.89 | 131,000 | Conforms - white to |
| | | 1.01 | | | off-white firm gel |
| | | | | | No Evidence of |
| | | | | | Crystal Growth |
| | 40° C. | 1.03 | 5.70 | 133,500 | Conforms - white to |
| | | 1.02 | | | off-white firm gel |
| | | | | | No Evidence of |
| | | | | | Crystal Growth |
| Cream C | 25° C. | 0.89 | 4.68 | 595,000 | Conforms - white to |
| | | 1.09 | | | off-white homogeneous |
| | | | | | cream |
| | 40° C. | 1.12 | 4.61 | 580,000 | Conforms - white to |
| | | 0.94 | | | off-white homogeneous |
| | | | | | cream |

¹Listed in order from top and bottom. No New impurities were observed. All levels of isomer impurity were consistent with API at ~2.2 area %

Next, we evaluated rapamycin crystal growth by microscopic examination of each of the gel formulations following 1-month storage at 5° C., 25° C., or 40° C. Gels 1A and 1B showed evidence of crystal growth at the higher temperatures, as shown in Table 11.

TABLE 11
Microscopic observations +/− of rapamycin
crystal growth in base vehicles

| | Evidence of | Evidence of | Evidence of | |
|---|---|---|---|---|
| | crystal | crystal | crystal | |
| Formula- | growth | growth | growth | |
| tion | at 5° C.? | at 25° C.? | at 40° C.? | Comments |
| Gel 1A | No | Yes | Yes | DMI/HEC |
| Gel 1B | No | Yes | Yes | TC/HEC |
| Gel 1C | No | No | No | PG/HEC |
| Gel 2A | No | No | No | DMI/PAA |
| Gel 2B | No | No | No | TC/PAA |
| Gel 2C | No | No | No | PG/PAA |

DMI: Dimethyl isosorbide, TC: Transcutol, PG: Propylene Glycol, HEC: Hydroxyethyl cellulose, PAA: poly(acrylic acid), tradename Carbomer.

Evidence of rapamycin crystal growth in the range of 25-100 microns (length) was observed as rectangular crystals and a depletion of the fine particles in the field. No crystals were observed in reserved vehicle formulations, including those exposed to freeze-thaw cycles. Since no crystals were seen in the vehicle samples, this did not appear to be an excipient issue (i.e., precipitation of an excipient). Gel 1B had the greatest amount of crystal formation and was eliminated from further analyses.

The remaining five formulations were further tested for rapamycin stability after 3 months of storage at 25° C. or 40° C. (Table 12); as well as for crystal growth after 3 months storage at 5° C., 25° C. and 40° C. (Table 13). None of the formulations showed crystal growth at 5° C., but after 3 months of storage at 25° C. or 40° C., Gel 1C was the only formulation that did not show crystal growth.

TABLE 12
Three-month stability results for the remaining
4 prototype 1.0% rapamycin formulations.

| | | Assay | | Viscosity | |
|---|---|---|---|---|---|
| Formulation | Temp. | % w/w¹ | pH | cP | Appearance |
| Gel 1C | 25° C. | 0.99 | 4.66 | 26,100 | Conforms - white to |
| | | 1.02 | | | off-white soft gel |
| | | | | | No Evidence of |
| | | | | | Crystal Growth |
| | 40° C. | 1.02 | 4.64 | 25,800 | Conforms - white to |
| | | 1.03 | | | off-white soft gel |
| | | | | | No Evidence of |
| | | | | | Crystal Growth |
| Gel 2A | 25° C. | 1.04 | 5.70 | 120,000 | Conforms - white to |
| | | 1.03 | | | off-white firm gel |
| | | | | | Evidence of Crystal |
| | | | | | Growth |
| | 40° C. | 1.03 | 5.64 | 121,300 | Conforms - white to |
| | | 1.00 | | | off-white firm gel |
| | | | | | Evidence of Crystal |
| | | | | | Growth |
| Gel 2B | 25° C. | 1.03 | 4.71 | 126,200 | Conforms - white to |
| | | 1.01 | | | off-white firm gel |
| | | | | | Evidence of Crystal |
| | | | | | Growth |
| | 40° C. | 0.99 | 5.70 | 123,600 | Conforms - white to |
| | | 1.01 | | | off-white firm gel |
| | | | | | Evidence of Crystal |
| | | | | | Growth |
| Gel 2C | 25° C. | 1.01 | 5.75 | 128,400 | Conforms - white to |
| | | 1.01 | | | off-white firm gel |
| | | | | | Evidence of Crystal |
| | | | | | Growth² |
| | 40° C. | 1.00 | 5.74 | 1,217,900 | Conforms - white to |
| | | 0.99 | | | off-white firm gel |
| | | | | | Evidence of Crystal |
| | | | | | Growth² |

¹Listed in order from top and bottom. No New impurities were observed. All levels of isomer impurity were consistent with API at ~2.2 area %
²Evidence of Crystal growth observed at 2-months

TABLE 13
Rapamycin crystal growth following 3 months at
5° C., 25° C. or 40° C.

| | Evidence of | Evidence of | Evidence of | |
|---|---|---|---|---|
| | crystal | crystal | crystal | |
| Formula- | growth at | growth at | growth at | |
| tion | 5° C.? | 25° C.? | 40° C.? | Comments |
| Gel 1A | No | Yes* | Yes* | DMI/HEC/pH 4.5 |
| Gel 1B | No | Yes* | Yes* | TC/HEC/pH 4.5 |
| Gel 1C | No | No | No | PG/HEC/pH 4.5 |
| Gel 2A | No | Yes | Yes | DMI/PAA/pH 6 |
| Gel 2B | No | Yes | Yes | TC/PAA/pH 6 |
| Gel 2C | No | Yes** | Yes** | PG/PAA/pH 6 |

DMI: Dimethyl isosorbide. TC: Transcutol, PG: Propylene Glycol, HEC: Hydroxyethyl cellulose, PAA: poly(acrylic acid), tradename Carbomer.
*This was observed at the 1 month time point
**This was observed at the 2 month time point.

### Demonstration Rapamycin Batches of Gel 1C

Batches of different concentrations of micronized rapamycin, 0.1%, 1.0% and 2.0% w/w, were prepared in Gel 1C (PG/HEC). These development batches were manufactured at a 2.0 kg scale based on the processed described above for the prototype batches. Batch mixing was performed with a Lightnin' Mixer and an ultra Turrex homogenizer. The demonstration batches were prepared using sodium citrate/citric acid to maintain an acidic pH in the range of pH 3-5 and contained BHA as the antioxidant. Other excipients in included benzyl alcohol as a preservative, PS80, and EDTA. The bulk drug product from the development batches were filled into three tube types: 1) laminate blind end tube (item #7347); laminate nasal tip tube (item #7336); and epoxy-coated aluminum blind end tube (item #7343). Analytical testing of the finished products for three demonstration batches of 0.1%, 1.0% and 2.0% rapamycin showed that each of the batches was satisfactory in terms of its physical appearance as a white to off-white gel free of foreign particles, the amount of the API (rapamycin), the API particle size assayed as number of particles greater than 50 micron per 5 mg of sample, pH, viscosity, and amounts of benzyl alcohol and BHA.

The laminate blind-end tube and the epoxy-coated aluminum tubes for the three rapamycin concentrations were monitored during for stability at 5° C., 25° C. and 40° C. The 1-month analysis of the three concentrations of rapamycin in the laminate blind end tubes is summarized in Table 14 and the 6-month data stability results for the three different rapamycin concentration formulations is provided in Table 15.

TABLE 14
One-month stability data of rapamycin contained in laminate blind-end tubes.

| | | | rapamy- | | |
|---|---|---|---|---|---|
| | | | cin | | |
| | | | Concen- | | |
| Test | Specification | | tration | Temp. | Result |
| Assay¹ | 90-110% | 0.10% (0.0900- | 0.10% | 5° C. | 93.8% |
| Rapamycin | Label | 0.1100% w/w) | PD18017 | 25° C. | 95.1% |
| | Claim | | | 40° C. | 93.7% |
| | | 1.00% (0.900- | 1.0% | 5° C. | 93.8% |
| | | 1.100% w/w) | PD18018 | 25° C. | 94.8% |
| | | | | 40° C. | 94.0% |
| | | 2.00% (1.800- | 2.0% | 5° C. | 93.6% |
| | | 2.200% w/w) | PD18019 | 25° C. | 94.4% |
| | | | | 40° C. | 95.1% |
| Assay | Report Results | | 0.10% | 5° C. | 98% |
| Benzyl | (Claim: 1.00%) | | PD18017 | 25° C. | 98% |
| Alcohol | | | | 40° C. | 98% |
| | | | 1.0% | 5° C. | 97% |
| | | | PD18018 | 25° C. | 97% |
| | | | | 40° C. | 97% |
| | | | 2.0% | 5° C. | 93% |
| | | | PD18019 | 25° C. | 92% |
| | | | | 40° C. | 92% |
| Assay | Report Results | | 0.10% | 5° C. | 90% |
| Butylated | (Claim: 0.01% w/w) | | PD18017 | 25° C. | 87% |
| Hydroxyanisole | | | | 40° C. | 81% |
| | | | 1.0% | 5° C. | 92% |
| | | | PD18018 | 25° C. | 88% |
| | | | | 40° C. | 83% |
| | | | 2.0% | 5° C. | 94% |
| | | | PD18019 | 25° C. | 89% |
| | | | | 40° C. | 81% |

TABLE 15
Six-month rapamycin stability data of demonstration batches stored in epoxy-coated aluminum tubes.

| | | Stability Data - Assay¹ | | | | | | | | | | | |
|---|---|---|---|---|---|---|---|---|---|---|---|---|---|
| | | 1 Month | | | 2 Month | | | 3 Month | | | 6 Month | | |
| | Condi- | | Benzyl | | | Benzyl | | | Benzyl | | | Benzyl | |
| Sample | tion | Rapamycin² | Alcohol | BHA | Rapamycin² | Alcohol | BHA | Rapamycin² | Alcohol | BHA | Rapamycin² | Alcohol | BHA |
| 0.1% | 5° C. | 97.5% | 96% | 100% | 95.7% | 104% | 96% | 98.4% | 98% | 98% | 95.9% | 97% | 95% |
| PD18017 | 25° C. | 98.7% | 98% | 100% | 95.8% | 104% | 96% | 97.6% | 97% | 98% | 94.4% | 97% | 95% |
| | 40° C. | 97.3% | 97% | 100% | 93.7% | 101% | 91% | 94.6% | 96% | 94% | NP | NP | NP |
| 1.0% | 5° C. | 95.8% | 96% | 97% | 95.6% | 102% | 97% | 99.2% | 98% | 100% | 95.6% | 97% | 97% |
| PD18018 | 25° C. | 96.7% | 98% | 98% | 95.4% | 103% | 95% | 96.5% | 97% | 98% | 95.0% | 97% | 95% |
| | 40° C. | 96.4% | 97% | 97% | 94.2% | 100% | 93% | 97.2% | 96% | 96% | NP | NP | NP |
| 2.0% | 5° C. | 96.8% | 93% | 97% | 98.6% | 99% | 97% | 98.5% | 92% | 102% | 95.5% | 92% | 97% |
| PD18019 | 25° C. | 98.7% | 93% | 97% | 96.7% | 99% | 96% | 98.1% | 91% | 99% | 95.% | 91% | 94% |
| | 40° C. | 97.2% | 93% | 96% | 95.8% | 97% | 90% | 97.4% | 91% | 97% | NP | NP | NP |

¹All Assays recorded as % w/w to label claim: Benzyl Alcohol = 1.0% w/w, BHA (Butylated hydroxyanisole): 0.01% w/w
²A rapamycin reference standard was not available during the manufacturing or the initial testing of the demonstration batches. The drug product was assayed against the input micronized rapamycin (Apotex SIR160397). The potency of this material was taken from the CoA of the unmicronized material which did not account for loss during milling. When micronized rapamycin lot SIR160397 was assayed against a reference standard (Apotex YT38-012, 99.3%) the potency of the micronized rapamycin was determined to be 94%.
NP = Not Performed

### Example 4. Tolerability and Skin Penetration of Gel Formulations

### 5-Day Dermal Toleration Study

The minipig was selected as a model system because of its suitability for testing skin medications. A 5-day study was performed to assess the maximum tolerated dose (MTD) of once-daily topical application to the skin of gel formulation 1C (“Gel 1C”). Treatments were vehicle only (0% rapamycin), and 0.3% 1%, and 2% rapamycin.

On the day prior to the first application of the test gels, the hair was clipped from the back and sides of the trunk of each animal. Further clipping took place as needed. Care was taken to maximize the time period between clippings and application in order to minimize interference due to mechanical stress of the skin. The untreated skin on the dorsal part of the thighs was used as a control. Two male Gottingen SPF minipigs were marked on the back in 8 areas measuring 3×3 cm (9 cm²). The test gel was applied once daily (every 24 hours) to each of two designated areas (replicates) per animal, for a period of 5 days, as shown in Table 16. The test gel was spread uniformly over the indicated areas and massaged gently into the skin. The application sites were rinsed only when visible gel residue was present the next morning prior to the next application. In those instances, rinsing was performed with gauze or a paper towel moistened with luke-warm water. The eight application sites were left uncovered. Temperature was maintained at 21° C.+/−3° C. The room was illuminated from 06:00 h to 18:00 h to yield a 12-hour light/dark cycle. Animals were fed twice a day and had ad libitum access to water.

TABLE 16
Treatment schedule

| | | Rapamycin |
|---|---|---|
| Animal | Treatment | concentration |
| Nos | site | (%) |
| 1-2 | A | 0 (vehicle) |
| (White, | B | 0.3 |
| Group 1) | C | 1 |
| | D | 2 |

Mortality, clinical signs, skin reactions, body weight, and food consumption were monitored. Dose volume (ml/kg body weight) was 0.75 ml/kg per test area of 10% of the skin surface area, equaling 19.5 mg/cm² (1 ml of test article corresponds to 1 g). Any dermal irritation was scored according to the OECD Guideline for Testing of Chemicals No. 404, adopted 28 Jul. 2015: “Acute Dermal Irritation/Corrosion.” Below are the scoring levels used to evaluate erythema and eschar formation.

| Erythema and Eschar Formation | Score |
|---|---|
| No erythema | 0 |
| Very slight erythema (barely | 1 |
| perceptible) | |
| Well defined erythema | 2 |
| Moderate to severe erythema | 3 |
| Severe erythema (beet redness) | 4 |
| to eschar formation preventing | |
| grading of erythema | |

| Edema Formation | Score |
|---|---|
| No edema | 0 |
| Very slight edema (barely | 1 |
| perceptible) | |
| Slight edema (edges of area well | 2 |
| defined by definite raising) | |
| Moderate edema (raised | 3 |
| approximately 1 mm) | |
| Severe edema (raised more than | 4 |
| 1 mm, extending beyond area | |
| of exposure) | |

Any other dermal reactions (e.g., desquamation, fissuring, scab, exfoliation, necrosis) were recorded and the area involved scored according to internal SOP procedures as follows:

| Size of area of other | |
|---|---|
| reactions | Score |
| No skin reactions | 0 |
| <10% of test area | 1 |
| 10-25% of test area | 2 |
| 26-50% of test area | 3 |
| 51-75% of test area | 4 |
| 76-100% of test area | 5 |

There were no deaths or animals sacrificed during the course of this study and no clinical signs were observed throughout the study period. No skin reactions were observed. There were no apparent effects on food consumption, except one animal lost weight (3%) during the Gel 1C application period; however, this was considered as incidental and not toxicologically relevant as the change was <10% and there was no associated decrease in food consumption or any aberrant clinical signs observed. The conclusion of the 5-day study indicated that following once-daily dermal applications of 0 (vehicle), 0.3. 1, and 2% rapamycin Gel 1C formulations on minipigs, no clinical observations, rapamycin-related dermal reactions or apparent effects on body weight or food consumption were noted, demonstrating that all three concentrations of rapamycin Gel 1C formulations as well as the vehicle gel formulation itself were well tolerated.

### Skin Penetration of Four Rapamycin Formulations

A single dermal dose ADME (absorption, distribution, metabolism, and excretion) study on minipigs was performed using four topical rapamycin formulations and a 1% rapamycin formulation in a Vanicream™ base. Five male Gottingen minipigs were used in the study and were maintained under standardized conditions for validated dermal ADME testing as required by the FDA for product testing. The four topical formulations tested in the study were designated Formulation 1 Gel 1C (PG/HEC), Formulation 2 Gel 2A (DMI/PAA), Formulation 3 Gel 2B (TC/PAA) and Formulation 4 Gel 2C (PG/PAA). In the study, all test rapamycin formulations were characterized as a white to off-white firm gel. The rapamycin formulation in the Vanicream base contained 1% rapamycin. The Vanicream base formulation was characterized as a white cream. All rapamycin formulations were stored at 5° C. The ADME study experimental design is shown in Table 17.

TABLE 17
Experimental design comparing four topical rapamycin formulations to a commercial
1% rapamycin Vanicream ™ formulation in minipigs.

| | | | Dose | Dose | | |
|---|---|---|---|---|---|---|
| Group | | Test | Volume | Concentration | Dose | Number |
| No. | Test Materials | Site | (ml) | (mg/mL) | (mg) | of Males |
| 1 | Rapamycin (Formulation 1 -Gel 1C) | 1 | 2 | 10 | 20 | 1 |
| | 1% Rapamycin in Vanicream ™ | 2 | 2 | 10 | 20 | |
| 2 | Rapamycin (Formulation 2-Gel 2A) | 1 | 2 | 10 | 20 | 1 |
| | 1% Rapamycin in Vanicream ™ | 2 | 2 | 10 | 20 | |
| 3 | Rapamycin (Formulation 3-Gel 2B) | 1 | 2 | 10 | 20 | 1 |
| | 1% Rapamycin in Vanicream ™ | 2 | 2 | 10 | 20 | |
| 4 | Rapamycin (Formulation 4-Gel 2C) | 1 | 2 | 10 | 20 | 1 |
| | 1% Rapamycin in Vanicream ™ | 2 | 2 | 10 | 20 | |

The rapamycin formulations were administered dermally twice daily every 12 hours+/−30 minutes by application directly to the skin in a uniform layer. All animals had intact skin. The dorsal surface was prepared by close clipping of the hair with a small animal clipper prior to the first application. Care was taken to avoid abrasion of the skin. Each site was approximately 5 cm×5 cm and was placed at approximately the same location on either side of the spine. Residual test material was removed (following 11 hours and 38 minutes to 11 hours and 51 minutes of exposure) by gently wiping the site with gauze soaked in reverse osmosis (RO) water, followed by dry gauze. The test sites were rinsed in the same manner prior to scheduled euthanasia. The first day of dosing was designated as Day 1.

Mortality/morbidity checks were performed twice daily. Cage-side observations were performed once daily during week 1 and on day 1; observations were performed 1 to 3 hours post-application on day 1. A detailed clinical observation was performed on the day of randomization. Individual body weights were recorded on the day of randomization and on day 1. Animals were euthanized by sodium pentobarbital injection on day 2 (12 hours+/−30 minutes following application of rapamycin gel formulation). Following euthanasia, 4 skin-punch biopsies were collected (two 8 mm biopsy samples of full thickness skin from each site) from each animal. Each biopsy sample was weighed and then frozen in liquid nitrogen. Biopsies were separated into skin layers (e.g., epidermis, dermis, etc.) and analyzed for rapamycin concentration using an ultra-high performance liquid chromatography with tandem mass spectrometry (UHPLC-MS/MS) method with a Shimadzu Nexera® UHPLC coupled with an Applied Biosystems/MDS Sciex API 5500 UHPLC-MS/MS system in the positive electron ionization (ES1+) mode. The method was calibrated over the concentration range of 1 to 5000 ng/mL (4 to 20,000 ng/g skin tissue) using a 0.2 mL extracted sample. Rapamycin and the internal standard (IS, ascomycin) were extracted from pig skin by protein precipitation and extraction with 1:1 (v:v) DMSO:ACN. ACN refers to acetonitrile. Statistical analysis including regression analysis and descriptive statistics including arithmetic means and standard deviations were done using Watson Laboratory Information Management System (LIMS) and Microsoft Excel.

For tissue separation, the frozen biopsy samples were thawed at room temperature. The subcutaneous adipose tissue and/or muscle, if present, were removed. The stratum corneum was removed from the punch biopsy by tape stripping using Blenderm™ up to 20 times. Tape was applied by rubbing onto the skin and immediately pulling off. This was done with care as not to remove the epidermis layer. All tapes used to remove the stratum corneum were maintained in an individual, uniquely labeled cryovial, and stored in a freezer set to maintain −70° C. The biopsy sample was then scraped to remove the epidermis layers from the dermis using a scalpel and forceps. The epidermis layers were collected and were placed in an individual, uniquely labeled cryovial, weighed, and stored in a freezer set to maintain −70° C. The remaining dermal layers were weighed, placed in an individual uniquely, labeled cryovial, and stored in a freezer set to maintain −70° C. Separate forceps and scalpels were used for each layer of tissue being sectioned. Forceps were cleaned with alcohol between processing each biopsy sample. The three separated layers, each in an individual, uniquely labeled cryovial, of each biopsy sample was shipped to the bioanalytical laboratory for analysis.

### Results

There were no clinical signs or early deaths noted in the study. The concentrations of rapamycin were measured in the intact full thickness biopsy punch and the separated skin layers (stratum corneum, epidermis, and dermis) of the full thickness biopsy punch. A summary of the rapamycin concentrations and the weights of the full biopsy samples is shown in Tables 18 and 19, respectively. Because the tape strip accounts for the majority of the weight used to determine the concentration of rapamycin in the stratum corneum, this concentration is not an accurate reflection of the actual concentration of rapamycin in this barrier layer of the skin. As such, the results should not be considered comparable to those obtained from the other layers of tissues assessed in this study.

TABLE 18
Summary of rapamycin concentrations (ng/g skin tissue) in biopsy samples in minipigs.

| Test | | Dose | Full | Stratum | | |
|---|---|---|---|---|---|---|
| Site | Formulation | (mg) | Biopsya | Corneum^{b,c} | Epidermis^{b} | Dermis^{b} |
| 1 | Rapamycin (Formulation 1-Gel IC) | 20 | 897000 | 1690000 | 77200000 | 16300 |
| 1 | Rapamycin (Formulation 2-Gel 2A) | 20 | 429000 | 3370 | 39600000 | 41000 |
| 1 | Rapamycin (Formulation 3-Gel 2B) | 20 | 29500 | 6970 | 14400000 | 30500 |
| 1 | Rapamycin (Formulation 4-Gel 2C) | 20 | 191000 | 2520 | 15800000 | 35700 |
| 2 | 1% Rapamycin in Vanicream ™ ^{d} | 20 | 14560 | 2251 | 746400 | 4018 |

^{a}Concentrations measured from the full thickness biopsy punch including the stratum corneum, epidermis, dermis, and subcutaneous fat.
^{b}Concentrations measured from separated skin layers.
^{c}Weight of tape strips used to collect the stratum corneum. The majority of the weight is attributed to the tape strip and the actual weight of the stratum corneum cannot be accurately determined.
^{d} Mean concentration calculated from 4 individual measurements.

TABLE 19
Weight (g skin tissue) of full biopsy tissue samples in minipigs.

| Test | | Full Biopsy | | |
|---|---|---|---|---|
| Site | Formulation | (Sample 1) | Epidermis | Dermis |
| 1 | Rapamycin | 0.322 | 0.0004 | 0.1076 |
| | (Formulation 1- | | | |
| | Gel 1C) | | | |
| 1 | Rapamycin | 0.218 | 0.0006 | 0.1204 |
| | (Formulation 2- | | | |
| | Gel 2A) | | | |
| 1 | Rapamycin | 0.403 | 0.0004 | 0.1123 |
| | (Formulation 3- | | | |
| | Gel 2B) | | | |
| 1 | Rapamycin | 0.324 | 0.0009 | 0.1432 |
| | (Formulation 4- | | | |
| | Gel 2C) | | | |
| 2 | 1% Rapamycin in | 0.301 | 0.0005 | 0.1032 |
| | Vanicream ™ | | | |
| 2 | 1% Rapamycin in | 0.281 | 0.0007 | 0.1277 |
| | Vanicream ™ | | | |
| 2 | 1% Rapamycin in | 0.300 | 0.0003 | 0.1101 |
| | Vanicream ™ | | | |
| 2 | 1% Rapamycin in | 0.291 | 0.0006 | 0.1364 |
| | Vanicream ™ | | | |

All test formulations resulted in measurable concentrations of rapamycin in both the epidermis and the dermis layers. All four rapamycin test formulations (Gel 1C, 2A-C) delivered significantly greater concentrations of rapamycin to all layers of the skin and the full thickness biopsy than the 1% rapamycin in Vanicream™ control. Formulation 2 (Gel 2A) delivered the highest amount of rapamycin to the dermis layer, approximately 10× the amount compared to the 1% rapamycin in Vanicream™ control. Formulation 1 (Gel IC) delivered the highest amount of rapamycin to the epidermis, approximately 2× the amount compared to Formulation 2 (Gel 2A) and approximately 100× the amount compared to the 1% rapamycin in Vanicream™ control. Formulation 3 (Gel 2B) and Formulation 4 (Gel 2C) delivered approximately equivalent amounts of rapamycin to the dermis and epidermis layers, respectively. Importantly, Formulations 3 and 4 delivered to the dermis approximately 10× the amount of rapamycin than the 1% rapamycin in Vanicream™ control and approximately 20× the amount to the epidermis than to the Vanicream™ control. The dermis is an important site of activity for rapamycin in the treatment of facial angiofibroma and based on these results, ranking the amount of rapamycin measured in the dermis, the order, from highest to lowest, for the formulations is: Gel 2A>Gel 2C>Gel 2B>Gel 1C.

Various embodiments of the invention are described above in the Detailed Description and Examples. While these descriptions directly disclose the above embodiments, it is understood that those skilled in the art may conceive of modifications and variations of the specific embodiments shown and described herein. Any such modifications and variations that fall within the disclosed descriptions and examples are intended to be included therein as well. Unless noted otherwise, it is understood that the words and phrases used in the specification and claims be given their ordinary and plain meanings to those of ordinary skill in the application art.

1. A gel composition for topical administration consisting of a stable suspension of rapamycin in a homogeneous mixture of a gel structure forming base, a solvent, an antioxidant, a buffering agent adapted to maintain an acidic pH of the composition, and one or more optional excipients selected from a surfactant, a humectant, a chelating agent, and a preservative.

2. The composition of claim 1, wherein the gel structure forming base is selected from hydroxyethyl cellulose (HEC) and poly(acrylic acid) (PAA).

3. The composition of claim 2, wherein the gel structure forming base is HEC.

4. The composition of claim 3, wherein the HEC is present in an amount of from 0.5 to 5% w/w, preferably from about 1-2% w/w, or 1-1.75% w/w, based on the total weight of the composition.

5. The composition of claim 2, wherein the gel structure base is PAA.

6. The composition of claim 5, wherein the PAA is present in an amount of from about 0.1 to 3% w/w, 0.1 to 2.25% w/w, or 0.25 to 0.75% w/w, based on the total weight of the composition.

7. The composition of claim 1, wherein the composition comprises a solvent selected from propylene glycol (PG), dimethyl isosorbide (DMI), and diethylene glycol monoethylether (Transcutol® or TC).

8. The composition of claim 7, wherein the solvent is PG present in an amount of from about 5-25% w/w, preferably about 10-15% w/w, based on the total weight of the composition.

9. The composition of claim 7, wherein the solvent is DMI or TC.

10. The composition of claim 9, wherein the solvent is present in an amount of from about 5-25% w/w, preferably 6-8% w/w, based on the total weight of the composition.

11. The composition of claim 1, wherein the antioxidant is butylated hydroxyanisol (BHA).

12. The composition of claim 1, comprising a surfactant.

13. The composition of claim 12, wherein the surfactant is selected from the group consisting of polysorbate 80, polysorbate 60, polysorbate 40, polysorbate 20, PEG-40 stearate, steareth-20, steareth-100, ceteth-20, ceteareth-20, and sodium lauryl sulfate.

14. The composition of claim 12, wherein the surfactant is polysorbate 80.

15. The composition of claim 13, wherein the surfactant is present in an amount of from about 0.005 to 1% w/w, preferably 0.01 to 0.10% w/w, based on the total weight of the composition.

16. The composition of claim 1, comprising a preservative.

17. The composition of claim 16, wherein the preservative is benzyl alcohol.

18. The composition of claim 17, wherein the benzyl alcohol is present in an amount of from about 0.5% to 3% w/w, preferably 0.5% to 1.5% w/w, based on the total weight of the composition.

19. The composition of claim 1, wherein the rapamycin is present in an amount of from about 0.05% w/w to 2.0% w/w, based on the total weight of the composition.

20. The composition of claim 19, wherein the rapamcyin is present in an amount of about 0.10%, 1.0%, or 2.0% w/w, based on the total weight of the composition.

21. The composition of claim 1, wherein the rapamycin is micronized rapamycin.

22. The composition of claim 21, wherein the micronized rapamycin consists of micronized particles of rapamycin having a particle size distribution (PSD) defined by a D50 in the range of 1-5 microns.

23. The composition of claim 22, wherein the PSD is further defined by a D10 in the range of 1-2 microns and a D90 in the range of 4-8 microns.

24. The composition of claim 19, wherein the composition comprises hydroxyethyl cellulose (HEC) as the gel structure forming base, dimethyl isosorbide (DMI) as the solvent, and butylated hydroxyanisol (BHA) as the antioxidant.

25. The composition of claim 19, wherein the composition comprises hydroxyethyl cellulose (HEC) as the gel structure forming base, TC as the solvent, and butylated hydroxyanisol (BHA) as the antioxidant.

26. The composition of claim 19, wherein the composition comprises hydroxyethyl cellulose (HEC) as the gel structure forming base, propylene glycol (PG) as the solvent, and butylated hydroxyanisol (BHA) as the antioxidant.

27. The composition of claim 21, wherein the composition comprises poly(acrylic acid) as the gel structure forming base, dimethyl isosorbide (DMI) as the solvent, and butylated hydroxyanisol (BHA) as the antioxidant.

28. The composition of claim 21, wherein the composition comprises poly(acrylic acid) as the gel structure forming base, diethylene glycol monoethylether (TC) as the solvent, and butylated hydroxyanisol (BHA) as the antioxidant.

29. The composition of claim 21, wherein the composition comprises poly(acrylic acid) as the gel structure forming base, propylene glycol as the solvent, and butylated hydroxyanisol (BHA) as the antioxidant.

30. The composition of claim 24, wherein the composition further comprises from 0.025 to 0.25% w/w polysorbate 80 and 0.5 to 3.0% w/w benzyl alcohol.

31. The composition of claim 24, wherein the pH of the composition is less than 7.0, most preferably in the range of pH 3-6.

32. The composition of claim 31, wherein the rapamycin of the composition is stable against chemical degradation and physically stable against crystal growth for at least three months at 5 C.

33. The composition of claim 26, wherein the composition is formulated with an acidic pH, preferably a pH less than 7.0, most preferably in the range of pH 3-6, and the composition is stable against chemical degradation and physically stable against crystal growth for at least one month at 25 C or 40 C, optionally wherein the composition further comprises from 0.025 to 0.25% w/w polysorbate 80 and 0.5 to 3.0% w/w benzyl alcohol.

34. The composition of claim 26, wherein the composition is formulated with an acidic pH, preferably a pH less than 7.0, most preferably in the range of pH 3-6, and the composition is stable against chemical degradation and physically stable against crystal growth for at least three months at 25 C or 40 C and at least six months at 5 C or 25 C, optionally wherein the composition further comprises from 0.025 to 0.25% w/w polysorbate 80 and 0.5 to 3.0% w/w benzyl alcohol.

35. The composition of claim 1, for use in therapy.

36. The composition of claim 1 for use in a method of treating a skin condition, disease, or disorder.

37. The composition of claim 36, wherein the skin condition, disease, or disorder is selected from the group consisting of Acanthosis nigricans, acne, actinic keratosis, allergic conjunctivitis, ameloonychohypohidrotic syndrome, angiokeratoma, angiokeratomas in Fabry disease, angiomas including cherry angioma, senile angioma, spider angioma, strawberry angioma, and tufted angioma, athlete's foot, atopic dermatitis, bacterial vaginosis, balanitis, Bannayan-Riley-Ruvalcaba Syndrome, basal cell carcinoma, basal cell nevus Syndrome, Birt-Hogg-Dube Syndrome, blisters, blue rubber bleb nevus syndrome, bromhidrosis, Brook-Speigler Syndrome, bullous pemphigoid, calluses, candidiasis, carbunculosis, cavernous lymphangioma, cellulitis, cerebral atrophy-associated skin conditions, chelitis granulomatosis, Conradi-Eltinermann disease, Corneodermatoosseous syndrome-associated skin conditions, Cowden disease, cutaneous Castleman disease, cutaneous larva migrans, cutaneous sarcoidosis, cutaneous T-cell lymphoma (CTCL), decubitous ulcer, dermal atrophy incident to aging or senescence, dermatitis including contact dermatitis, drug-induced dermatitis, allergic dermatitis, nummular dermatitis, perioral dermatitis, neurodermatitis, seborrheic dermatitis, and atopic dermatitis, dermatofibrosarcoma protruberans, dermatophytosis, diffuse microcystic lymphatic malformations, discoid lupus erythematosus, dyshydrotic eczema, dyskeratosis congenita, ecthyma, eczema, epidermodysplasia verruciformis, epidermolysis bullosa simplex, epidermolytic ichthyosis, epithelial nevus including verrucous nevus, systematized nevus, inflammatory linear verrucous epidermal nevus, and sebaceous nevus, erysipalus, erythema multiforme, erythrokeratoderma variabilis, extramammary Paget's disease, familial cylindromatosis, familial multiple discoid fibromas, filariasis, focal acral hyperkeratosis, follicular hyperkeratosis, follicular hyperkeratosis associated with pilodental dysplasia with refractive errors, furunculosis, genital warts, gingival hypertrophy, granuloma, Hailey-Hailey disease, hemangioma simplex, hereditary footpad hyperkeratosis as afflicting dogs, Herpes, hives, hidradenitis suppurativa, hyperhidrosis, hyperkeratosis lenticularis perstans, hypomelanotic macules, ichthyosis hystrix, impetigo, incontinentia pigmenti, infantile hemangiomas, insect bites, juvenile polyposis syndrome, Kaposi sarcoma, Kaposiform hemangioendothlioma, keloid, microcystic lymphatic malformation, keloid scar disease, keratosis follicularis dwarfism-associated skin conditions, keratosis pilaris, KID syndrome, Klippel-Trenaunay syndrome, lentigines or liver spots, Lhermitte-Duclos syndrome, lichen planopilaris, lichenoid keratosis including lichen planus, lichen sclerosus, chronic erosive oral lichen, lupus, lymphangioma circumscriptum, melanoma, Merkel cell carcinoma, metastatic melanoma, microcystic lymphatic malformation, miliaria or heat rash, Milker's nodule, Molluscum contagiosum, Muir-Torre syndrome, multiple minute digitate hyperkeratosis, myiasis including furuncular myiasis and migratory myiasis, Netherton syndrome, skin and dermal manifestations of neurofibromatosis type 1 (also referred to as “NF1” or von Recklinhausen's Disease), nevus araneus, nonmelanoma skin cancer, Olmsted syndrome, onychomycosis tinea including tinea alba, tinea pedis, tinea unguium, tinea manuum, tinea cruris, tinea corporis, tinea capitis, tinea faciei, tinea barbae, tinea imbricata, tinea nigra, tinea versicolor, tinea incognito, oral lichen planus, oral mucosal disease due to GVHD, overgrowth syndromes, pachyonychia congenita, panniculitis, paronychia, pediculosis, pemphigoid disease, pemphigus vulgaris, periungual and subungual fibroma, Peutz-Jeghers syndrome, photo-aging by UV radiation, pigmented macule, including for example nevus spilus and cafe au lait spots, pityriasis, plantar hyperkeratosis, proteus syndrome, proteus-like syndrome, pruritis vulvae, psoriasis, pyrogenic granuloma, refractory hemangioendotheliomas in Maffucci syndrome, Refsum disease, rosacea, Rosai-Dorfman disease, scabies, scleroderma, seborrheic keratosis, Sezary syndrome, Sjogren-Larsson Syndrome, squamous cell carcinoma, statis dermatitis, Sturge-Weber Syndrome, telangiectasias, trichoepithelioma, trichomoniasis, skin tumor manifestations of tuberous sclerosis, vaginal yeast infection, vascular malformations including port wine stains and lymphangiomas, vitiligo vulgaris, warts, xeroderma and xeroderma pigmentosum.

38. The composition of claim 37, wherein the skin condition, disease, or disorder is selected from the group consisting of Birt-Hogg-Dube Syndrome, cutaneous T-cell lymphoma (CTCL) dermal atrophy incident to aging or senescence, skin and dermal manifestations of neurofibromatosis type 1 (also referred to as “NF1” or von Recklinhausen's Disease), oral lichen planus, oral mucosal disease due to GVHD, pachyonychia congenita, Sturge-Weber Syndrome, vascular malformations including port wine stains and lymphangiomas.

39. The composition of claim 36, wherein the skin condition, disease or disorder is selected from an angiofibroma, hemangioma, a vascular malformation, a pyogenic granuloma, essential telangiectasias, familial multiple discoid fibroma, and cherry angioma.

40. The composition of claim 39, wherein the skin condition, disease or disorder is a facial angiofibroma.

41. A method for treating a skin condition, disease or disorder in a human subject in need of such treatment, the method comprising applying the topical composition of claim 1 to the affected areas of the subject's skin in an amount suitable to cover the affected area with a thin layer of the composition.

42. The method of claim 41, wherein the skin condition, disease, or disorder is selected from the group consisting of Acanthosis nigricans, acne, actinic keratosis, allergic conjunctivitis, ameloonychohypohidrotic syndrome, angiokeratoma, angiokeratomas in Fabry disease, angiomas including cherry angioma, senile angioma, spider angioma, strawberry angioma, and tufted angioma, athlete's foot, atopic dermatitis, bacterial vaginosis, balanitis, Bannayan-Riley-Ruvalcaba Syndrome, basal cell carcinoma, basal cell nevus Syndrome, Birt-Hogg-Dube Syndrome, blisters, blue rubber bleb nevus syndrome, bromhidrosis, Brook-Speigler Syndrome, bullous pemphigoid, calluses, candidiasis, carbunculosis, cavernous lymphangioma, cellulitis, cerebral atrophy-associated skin conditions, chelitis granulomatosis, Conradi-Eltinermann disease, Corneodermatoosseous syndrome-associated skin conditions, Cowden disease, cutaneous Castleman disease, cutaneous larva migrans, cutaneous sarcoidosis, cutaneous T-cell lymphoma (CTCL), decubitous ulcer, dermal atrophy incident to aging or senescence, dermatitis including contact dermatitis, drug-induced dermatitis, allergic dermatitis, nummular dermatitis, perioral dermatitis, neurodermatitis, seborrheic dermatitis, and atopic dermatitis, dermatofibrosarcoma protruberans, dermatophytosis, diffuse microcystic lymphatic malformations, discoid lupus erythematosus, dyshydrotic eczema, dyskeratosis congenita, ecthyma, eczema, epidermodysplasia verruciformis, epidermolysis bullosa simplex, epidermolytic ichthyosis, epithelial nevus including verrucous nevus, systematized nevus, inflammatory linear verrucous epidermal nevus, and sebaceous nevus, erysipalus, erythema multiforme, erythrokeratoderma variabilis, extramammary Paget's disease, familial cylindromatosis, familial multiple discoid fibromas, filariasis, focal acral hyperkeratosis, follicular hyperkeratosis, follicular hyperkeratosis associated with pilodental dysplasia with refractive errors, furunculosis, genital warts, gingival hypertrophy, granuloma, Hailey-Hailey disease, hemangioma simplex, hereditary footpad hyperkeratosis as afflicting dogs, Herpes, hives, hidradenitis suppurativa, hyperhidrosis, hyperkeratosis lenticularis perstans, hypomelanotic macules, ichthyosis hystrix, impetigo, incontinentia pigmenti, infantile hemangiomas, insect bites, juvenile polyposis syndrome, Kaposi sarcoma, Kaposiform hemangioendothlioma, keloid, microcystic lymphatic malformation, keloid scar disease, keratosis follicularis dwarfism-associated skin conditions, keratosis pilaris, KID syndrome, Klippel-Trenaunay syndrome, lentigines or liver spots, Lhermitte-Duclos syndrome, lichen planopilaris, lichenoid keratosis including lichen planus, lichen sclerosus, chronic erosive oral lichen, lupus, lymphangioma circumscriptum, melanoma, Merkel cell carcinoma, metastatic melanoma, microcystic lymphatic malformation, miliaria or heat rash, Milker's nodule, Molluscum contagiosum, Muir-Torre syndrome, multiple minute digitate hyperkeratosis, myiasis including furuncular myiasis and migratory myiasis, Netherton syndrome, skin and dermal manifestations of neurofibromatosis type 1 (also referred to as “NF1” or von Recklinhausen's Disease), nevus araneus, nonmelanoma skin cancer, Olmsted syndrome, onychomycosis tinea including tinea alba, tinea pedis, tinea unguium, tinea manuum, tinea cruris, tinea corporis, tinea capitis, tinea faciei, tinea barbae, tinea imbricata, tinea nigra, tinea versicolor, tinea incognito, oral lichen planus, oral mucosal disease due to GVHD, overgrowth syndromes, pachyonychia congenita, panniculitis, paronychia, pediculosis, pemphigoid disease, pemphigus vulgaris, periungual and subungual fibroma, Peutz-Jeghers syndrome, photo-aging by UV radiation, pigmented macule, including for example nevus spilus and cafe au lait spots, pityriasis, plantar hyperkeratosis, proteus syndrome, proteus-like syndrome, pruritis vulvae, psoriasis, pyrogenic granuloma, refractory hemangioendotheliomas in Maffucci syndrome, Refsum disease, rosacea, Rosai-Dorfman disease, scabies, scleroderma, seborrheic keratosis, Sezary syndrome, Sjogren-Larsson Syndrome, squamous cell carcinoma, statis dermatitis, Sturge-Weber Syndrome, telangiectasias, trichoepithelioma, trichomoniasis, skin tumor manifestations of tuberous sclerosis, vaginal yeast infection, vascular malformations including port wine stains and lymphangiomas, vitiligo vulgaris, warts, xeroderma and xeroderma pigmentosum.

43. The method of claim 42, wherein the skin condition, disease, or disorder is selected from the group consisting of Birt-Hogg-Dube Syndrome, cutaneous T-cell lymphoma (CTCL) dermal atrophy incident to aging or senescence, skin and dermal manifestations of neurofibromatosis type 1 (also referred to as “NF1” or von Recklinhausen's Disease), oral lichen planus, oral mucosal disease due to GVHD, pachyonychia congenita, Sturge-Weber Syndrome, vascular malformations including port wine stains and lymphangiomas.

44. A method for treating facial angiofibromas in a human subject in need of such treatment, the method comprising applying the topical composition of claim 1 to the affected areas of the subject's skin in an amount suitable to cover the affected area with a thin layer of the composition.

45. A process for making the composition of claim 1, the process comprising
preparing a solvent phase in a first container by dissolving the antioxidant in the solvent followed by adding the gel base under continuous mixing,
preparing an aqueous phase in a second container by dissolving in water the surfactant, the preservative, the buffering agent, and any optional excipients,
dispersing the micronized rapamycin into the aqueous phase under continuous mixing,
subjecting the aqueous phase to high shear homogenization, and
combining the solvent phase with the aqueous phase under continuous mixing until the solvent and aqueous phases form a homogeneous gel composition.

46. An article of manufacture or package comprising the composition of claim 1.

47. The article or package of claim 47, wherein the composition is contained in a sealed or sealable epoxy-coated aluminum tube.
